(19) United States
(12) Patent Application Publication
(10) Pub. No.: US 2004/0029775 A1
(43) Pub. Date: Feb. 12, 2004

(54) Methods and compounds for influencing beta3-integrin-dependent intracellular processes

(76) Inventors: Meinrad Gawaz, Munchen (DE); Martin Ungerer, Munchen (DE); Waldemar Kolanus, Bonn (DE)

Correspondence Address: MYERS BIGEL SIBLEY & SAJOVEC, PO BOX 37428, RALEIGH, NC, 27627, US

(21) Appl. No.: 10/297,570

(22) PCT Filed: Jun. 11, 2001
(86) PCT No.: PCT/EP01/06588

(30) Foreign Application Priority Data
Jun. 9, 2000 (DE) 100-28-645.3
Jul. 28, 2000 (DE) 100-37-272.4

(51) Int. Cl. A61K 38/17; G01N 33/53

(52) U.S. Cl. 514/2; 435/7.1

(57) ABSTRACT

The invention relates to the use of β3-endonexin-long or β3-endonexin-short for finding active substances for the treatment of arteriosclerosis, unstable plaques resulting from the latter, acute coronary thrombosis, cardiac infarct, stroke, peripheral arterial occlusion diseases, chronic venous ulcer and restenosing processes.

The present invention relates to the influencing of β3-integrin-dependent intracellular processes for the purpose of treating acute and chronic vascular diseases. The present invention relates especially to new uses of the known proteins β3-endonexin-short and β3-endonexin-long to find active substances, and to specific methods of identifying active substances for the treatment of acute and chronic vascular diseases. The invention further relates to test mixtures for identifying active substances and to specific kits for identifying active substances. The invention relates also to medicaments that comprise active substances for influencing β3-integrin-dependent intracellular processes for the purpose of treating acute and chronic vascular diseases, and to methods of treatment that employ such medicaments.

Integrins are α/β heterodimeric surface proteins and belong to a class of adhesion receptors that mediate interactions between cells or between cells and the extracellular matrix.

β3-Integrins form a sub-class of the integrins and provide dynamic links between cells and the extracellular matrix, with information transfer between the following two key regions of β3-integrin being involved: the binding domains of the extracellular ligand and the intracellular cytoplasmic receptor domains. The signal transmission via β3-integrins is bidirectional. “Inside-out” signals regulate the affinity of β3-integrin for the ligand and control cell adhesion. “Outside-in” signals go from the β3-integrin connection to the extracellular matrix and regulate many fundamental intracellular processes, including cell survival and proliferation, cellular differentiation, morphogenesis, cell migration, phagocytosis, gene transcription and integrin localisation.

β3-Integrin is a constituent of the glycoprotein IIb/IIIa (GP IIb/IIIa, fibrinogen receptor) and of the vitronectin receptor. Glycoprotein IIb/IIIa is significant for the function of thrombocytes, especially activated thrombocytes, and plays an important role in acute cardiovascular diseases, such as, for example, acute coronary thrombosis. The vitronectin receptor is involved in the adhesion and migration of endothelial cells and plays an important role in chronic cardiovascular diseases, such as, for example, endothelial activation in the case of atherosclerosis or in unstable plaques.

β3-Integrins also play an important role in haemostasis, thrombosis, wound healing and angiogenesis and are highly expressed by many vascular cells, for example blood platelets, neutrophils, endothelial cells and cells of the smooth musculature. β3-Integrins show themselves to be modulators of a number of cellular functions, for example modulators of cell growth, intracellular signal transmission mechanisms and gene regulation.

It is known from the prior art to antagonise ligand binding to proteins of the receptor family containing β3-integrin. There are already several medicaments, so-called “GPIIb/IIIa antagonists”, such as, for example, Abciximab, RGD-peptides, fibans and fibatides, that are employed in the treatment of acute and chronic coronary syndromes. The effectiveness of such known GPIIb/IIIa antagonists is limited, however, by the fact that, after the existing receptors have been blocked, further receptor proteins from intracellular stores externalise to the cell membrane. On the other hand, the administered dose of such GPIIb/IIIa antagonists can be increased only within narrow limits since, in high concentrations, GPIIb/IIIa antagonists may lead to serious side-effects. Control of the externalisation of receptor proteins of the receptor family containing β3-integrin is not known from the prior art.

It is therefore an object of the invention to provide agents that permit the influencing of β3-integrin-dependent intracellular processes, and that accordingly

make it possible to stabilise or improve the activity of GPIIb/IIIa antagonists,

and make it possible to treat diseases the origin and/or course of which are connected with β3-integrin-dependent intracellular processes and that can be treated by regulation (inhibition or promotion) of β3-integrin-dependent intracellular processes.

A further object of the invention is to provide new uses of specific proteins in finding active substances for the treatment of vascular diseases.

It is a further object of the present invention to provide methods by which active substances can be found that are suitable for use in combination with known GPIIb/IIIa antagonists and that ensure or increase the effectiveness thereof.

It is a further object of the invention to provide medicaments that comprise identified active substances, and methods for the treatment of acute and chronic vascular diseases.

Those objects are achieved in accordance with the claims. The invention is based on the particular technical feature of influencing β3-integrin-dependent intracellular processes for the purpose of treating acute and chronic vascular diseases by the antagonistic activities of the known proteins β3-endonexin-short and β3-endonexin-long (Genbank accession No. HS371391). The proteins are known, for example, from Shattil S. J., et al., J. Cell Biology, 131, (1995), 807-816 and from Genbank, No. U37139 of May 12, 1995. In particular, the invention is based on the fact that, surprisingly, the known proteins β3-endonexin-short and β3-endonexin-long specifically control the internalisation and signal transmission of GPIIIb/IIa and vitronectin receptors. The internalisation is controlled by means of antagonisation of the activities of β3-endonexin-short and β3-endonexin-long, β3-endonexin-short promoting internalisation and the recycling of both receptors and β3-endonexin-long inhibiting internalisation of the receptors. In particular, it has been found that β3-endonexin-short specifically promotes the internalisation and the recycling of GPIIb/IIIa on human thrombocytes and of vitronectin receptors on human endothelial cells. In addition, various nuclear DNA transcription promoters for physiologically important mediators can be up- or down-regulated by means of β3-endonexin-short. On the other hand, it has been found that 3-endonexin-long, as an antagonist, blocks the internalisation of both receptors.

It has also been found that β3-endonexin-short and β3-endonexin-long are of great importance for intracellular signal transduction of vitronectin receptors in human endothelial cells, on the activation of endothelial cells, on the nuclear transcription of pro-atherogenic mediators and therefore in general for the process of atherogenesis and plaque destabilisation.

β3-Endonexin-short and β3-endonexin-long also regulate the surface expression or the secretion of signal substances that are of crucial importance for cardiovascular diseases (acute thrombosis, atherosclerosis, plaque rupture).

A peptide sequence motif conserved in both integrin receptors is crucial for the binding of β3-endonexin-short to both receptors. The binding specificity between β3-endonexin-short and the cytoplasmic domain of β3-integrin is due to a membrane-distal NITY motif within the cytoplasmic domain of β3-integrin. β3-Endonexin-long (20 kDa), on the other hand, inhibits the β3-endonexin-short-mediated integrin receptor internalisation.

The present invention offers the advantage that, by means of an intervention at β3-endonexin, it is possible to ensure or improve the activity of already existing medicaments. In particular, it is now possible both to influence, by existing medicaments (for example Abciximab, RGD-peptides, fibans, fibatides), the external ligand binding to GPIIb/IIIa and to vitronectin receptors and to influence, according to the invention, their internalisation-dependent intracellular effects. Advantageously, by controlling β3-endonexin-short and β3-endonexin-long, it is possible specifically to control the surface expression, internalisation and signal transduction of both receptors, β3-endonexin-short promoting internalisation whereas β3-endonexin-long blocks internalisation.

Proceeding from that realisation, not only is it possible, therefore, to ensure or improve the effectiveness of already existing medicaments but also it is now possible on the one hand specifically to influence the composition of the thrombocyte alpha-granules, that is to say their mediator reuptake capacity and their secretory properties. On the other hand, the activation of endothelial cells can likewise be influenced by endonexin by way of vitronectin receptor internalisation.

β3-Endonexin-short and β3-endonexin-long are therefore crucial signal transduction molecules and target proteins for the development of new pharmacological active substances in the case of acute and chronic vascular processes.

The present invention therefore provides a use of β3-endonexin-long or β3-endonexin-short for finding active substances for the treatment of arteriosclerosis, unstable plaques resulting from the latter, acute coronary thrombosis, cardiac infarct, stroke, peripheral arterial occlusion diseases, chronic venous ulcer and restenosing processes.

The present invention also provides a method of identifying compounds that inhibit binding of β3-endonexin-short to β3-integrin, which method is characterised by the following steps:

(a) incubation of a mixture comprising

(a1) β3-endonexin-short or a peptide or polypeptide that is functionally equivalent thereto with regard to binding to β3-integrin;

(a2) β3-integrin or a peptide or polypeptide that is functionally equivalent thereto with regard to binding to β3-endonexin-short;

(a3) a compound to be tested; and

(b) detection of the inhibition of the binding of component (a1) to component (a2) in the presence of compound (a3) in comparison with the absence of compound (a3).

The present invention also provides a method of identifying compounds that inhibit binding of β3-endonexin-long to β3-integrin, characterised by the following steps:

(a) incubation of a mixture comprising

(a1) β3-endonexin-long or a peptide or polypeptide that is functionally equivalent thereto with regard to its influence on β3-integrin;

(a2) β3-integrin or a peptide or polypeptide that is functionally equivalent thereto with regard to its interaction with β3-endonexin-long;

(a3) a compound to be tested; and

(b) detection of the inhibition of an influence on component (a2) by component (a1) in the presence of compound (a3) in comparison with the absence of compound (a3).

The influence by component (a1) on component (a2) can occur, in particular, as a result of direct or indirect binding.

The present invention also provides a method of identifying compounds that shift the competition of the binding of β3-integrin to β3-endonexin-short and β3-endonexin-long, characterised by the following steps:

(a) incubation of a mixture comprising

(a1) β3-endonexin-short or a peptide or polypeptide that is functionally equivalent thereto with regard to binding to β3-integrin;

(a2) β3-endonexin-long or a peptide or polypeptide that is functionally equivalent thereto with regard to binding to β3-integrin;

(a3) β3-integrin or a peptide or polypeptide that is functionally equivalent thereto with regard to binding to β3-endonexin-short and/or to β3-endonexin-long;

(a4) a compound to be tested; and

(b) detection of a shift in the ratio of the binding of component (a1) to component (a3) to the binding of component (a2) to component (a3) in comparison with the absence of the compound to be tested (a4).

The screening methods according to the invention can advantageously be carried out as high-throughput-screening (HTS) methods.

HTS-suitable screening tests on new “small molecules” that inhibit β3-endonexin-short can be carried out, for example, by testing small molecules from large substance libraries for their suitability to inhibit the binding of β3-endonexin-short to GPIIb/IIIa or to the vitronectin receptor.

On the one hand, one possible assay is an assay using individual protein components. On the other hand, an assay using CHO cells expressing GPIIb/IIIa and β3-endonexin-short, especially in a stable manner, would be possible. The type of assay is a matter of choice for the person skilled in the art, the following possibilities being disclosed by way of example.

The following assays using individual protein components may be mentioned in particular. In one embodiment, β3-endonexin-short is avidin-immobilised on microtitre plates after biotinylation. In another embodiment, β3-endonexin-short is applied with a His-tag to nickel plates. The specific binding site of β3-endonexin-short to both receptors is known (Eigenthaler, M. et al., J. Biol. Chem., 272, (1997), 7693-7698) and is coupled to fluorescein, preferably as a 10-mer peptide, for example via a cysteine bridge. That construct is incubated on the plate in the presence of various test molecules, then washed in order subsequently to quantify the amount of construct remaining on the basis of the residual fluorescence. In another embodiment, an assay can also be carried out with the aid of fluorescence resonance energy transfer (FRET). For that purpose, a fusion protein comprising β3-endonexin-short-green fluorescent protein (GFP) is immobilised on plates as described above. The above-mentioned construct (10-mer peptide+fluorescein) is incubated therewith on the plate. The binding of β3-endonexin-short to the indicator peptide can then be measured on the basis of a wavelength shift of the exciting blue light to the emission of red light since, in the case of true binding, a resonance energy transfer occurs highly specifically between fluorescein and GFP.

It is also possible to use for the purposes of the method according to the invention an affinity column containing substances that might be potential binding partners. Purified fusion proteins of β3-endonexin-short and GFP are accordingly applied to affinity columns containing substances that may be potential binding partners. After washing, the residual fluorescence is quantified.

A cell assay for finding inhibitors of β3-endonexin-short may specifically comprise the following steps:

(a) cultivation in conventional cell culture media of Chinese hamster ovary cells (CHO) expressing β3-integrin in a stable manner, prepared according to the instructions in Ylänne et al., J Biol Chem 1995; 270: 9550-9557;

(b) induction of an expression of β3-endonexin-short, preferably by liposomal gene transfer into those cells;

(c) addition of a compound to be tested to the culture medium of the cells;

(d) measurement of the surface density of the β3-integrins on the cells by means of fluorescence-activated cell sorting (FACS) using conventional specific antibodies after a certain time has elapsed, for example 24 hours later;

(e) detection of the increase in surface density in the presence of the substance from step (c) in comparison with the absence thereof.

Alternatively, as described in FIG. 5B, the constitutive internalisation of Texas-Red-conjugated anti-IgG1-MAK can be determined by means of a fluorescence technique. In this case, the GPIIb/IIIa internalisation (measurement by means of antibody binding and FACS) can be examined for inhibition by small molecules.

HTS-suitable screening tests on new small molecules that inhibit β3-endonexin-long can be carried out, for example, by testing small molecules from large substance banks for their suitability to inhibit the binding of long to GPIIb/IIIa or to the vitronectin receptor.

On the one hand, one possible assay is an assay using individual protein components. On the other hand, an assay using CHO cells expressing GPIIb/IIIa and β3-endonexin-long, especially in a stable manner, would be possible. The type of assay is a matter of choice for the person skilled in the art, the following possibilities being disclosed by way of example.

The following assays using individual protein components may be mentioned in particular. In one embodiment, aptamers or specific antibodies to the protein regions occurring only in β3-endonexin-long and not occurring in β3-endonexin-short are selected. For this, β3-endonexin-short and the protein domains occurring only in β3-endonexin-long are bacterially over-expressed and purified by means of a GST-tag. It is then possible, by means of NEB technology, to select specific aptamers or antibodies that bind only to that construct and not to purified β3-endonexin-short. Aptamers can then be converted by means of conversion technology into small molecules which are then used with the techniques described below to identify inhibitors.

In particular, β3-endonexin-long can be avidin-immobilised on microtitre plates after biotinylation. In another embodiment, β3-endonexin-long can be applied with a HIS-tag to nickel plates. β3-Integrin is coupled to fluorescein, for example by way of a cysteine bridge. That construct is incubated on the plate in the presence of various test molecules, then washed in order subsequently to quantify the amount of construct remaining on the basis of the residual fluorescence.

It is also possible, however, to carry out an assay with the aid of fluorescence resonance energy transfer (FRET). For that purpose, a fusion protein β3-endonexin-long-green fluorescent protein (GFP) is immobilised on plates as described above. The above-mentioned construct (β3-integrin+fluorescein) is incubated therewith on the plate. The binding of β3-endonexin-long to the integrin can then be measured on the basis of a wavelength shift of the exciting blue light to the emission of red light since, in the case of true binding, a resonance energy transfer occurs highly specifically between fluorescein and GFP.

In another embodiment, an affinity column containing substances that might be potential binding partners can be used. In particular, purified fusion proteins of β3-endonexin-long and GFP are applied to affinity columns containing substances that might be potential binding partners. After washing, the residual fluorescence is quantified.

A cell assay for finding inhibitors of β3-endonexin-long may specifically comprise the following steps:

(a) cultivation in conventional cell culture media of Chinese hamster ovary cells (CHO) expressing β3-integrin in a stable manner, prepared according to the instructions in Ylänne et al., J Biol Chem 1995; 270: 9550-9557;

(b) induction of an expression of β3-endonexin-long, preferably by liposomal gene transfer into those cells;

(c) addition of a compound to be tested to the culture medium of the cells;

(d) measurement of the surface density of the β3-integrins on the cells by means of fluorescence-activated cell sorting (FACS) using conventional specific antibodies after a certain time has elapsed, for example 24 hours later;

(e) detection of the decrease in surface density in the presence of the substance from step (c) in comparison with the absence thereof.

Alternatively, as described in FIG. 5B, the constitutive internalisation of Texas-Red-conjugated anti-IgG1-MAK can be determined by means of a fluorescence technique. In this case, the GPIIb/IIIa internalisation (measurement by means of antibody binding and FACS) can be examined for inhibition by small molecules.

The present invention also provides a test mixture for identifying compounds that inhibit binding of β3-endonexin-short or β3-endonexin-long to β3-integrin, characterised by the following components:

(a) a first component selected from the group consisting of β3-integrin and peptide or polypeptide that is functionally equivalent thereto with regard to binding to β3-endonexin-short and/or to β3-endonexin-long;

(b) a second component selected from a first and/or a second group, wherein

(b1) the first group consists of β3-endonexin-short and peptides or polypeptides that are functionally equivalent thereto with regard to binding to β3-integrin;

(b2) the second group consists of β3-endonexin-long and peptides or polypeptides that are functionally equivalent thereto with regard to binding to β3-integrin.

The present invention further provides a kit for identifying compounds that inhibit binding of β3-endonexin-short or β3-endonexin-long to β3-integrin, the kit being characterised by the following components:

(a) a first component selected from the group consisting of β3-integrin and peptide or polypeptide that is functionally equivalent thereto with regard to binding to β3-endonexin-short and/or to β3-endonexin-long;

(b) a second component selected from a first and/or a second group, wherein

(b1) the first group consists of β3-endonexin-short and peptides or polypeptides that are functionally equivalent thereto with regard to binding to β3-integrin;

(b2) the second group consists of β3-endonexin-long and peptides or polypeptides that are functionally equivalent thereto with regard to binding to β3-integrin.

The present invention also provides a medicament that comprises a compound for influencing β3-integrin-dependent intracellular processes, the compound being characterised in that, for promoting β3-integrin-internalisation-dependent processes and/or for inhibiting the secretion of pro-atherogenic mediators from endothelial cells and for inhibiting the expression of migration-promoting surface receptors on endothelial cells, it has an activating effect on β3-endonexin-short and/or an inhibiting effect on β3-endonexin-long, or, for inhibiting β3-integrin-internalisation-dependent processes and/or for inhibiting the secretion of pro-atherogenic mediators from thrombocytes, it has an activating effect on β3-endonexin-long and/or an inhibiting effect on β3-endonexin-short. That medicament can be employed, according to the active substance contained therein, for the treatment of acute and chronic vascular diseases, such as arteriosclerosis and unstable plaques resulting from the latter, acute coronary thrombosis and cardiac infarct, stroke, peripheral arterial occlusion disease, chronic venous ulcer and restenosing processes following surgical intervention in the area of the coronary arteries, cerebral arteries and peripheral arteries. That medicament can also be used prophylactically for the prevention of the development of atherogenic syndromes, such as coronary artery stenosis and stenoses in the area of the cerebral or peripheral arteries.

It has surprisingly been found that β3-endonexin regulates the surface expression of β3-integrin and β3-integrin-dependent intracellular processes and that this can be influenced by means of β3-endonexin. In particular, it is possible to influence the internalisation of β3-integrin, the surface recycling of β3-integrin and the composition of thrombocytic and endothelial storage vesicles. Furthermore, an influence can be exerted by means of β3-endonexin on the activation of promoters for surface receptor genes in endothelial cells.

In the experiments leading to the present invention, single-chained chimeras were produced with variants and mutated forms of all cytoplasmic β3-domains. After transient transfection in CHO cells or primary cultures of HUVEC cells, it was found that the β3-A chimeras exhibited a greatly reduced cell surface expression in comparison with the corresponding β3-B or β3-C fusion proteins or tailless constructs, while the “steady-state” levels of all the chimeras were virtually identical. The investigations using mutants of the cytoplasmic domain also showed that the NITY motif in the case of a3-A (Pos. 756-759) is of critical importance for the plasma expression of β3-A. In addition, the transport of β3-A to the cell surface was specifically modulated by the cytoplasmic protein β3-endonexin. β3-Endonexins occur as two different splice variants that are characterised by differing ability with regard to interaction with the β3 tail. The coexpression of the native long form of β3-endonexin (β3-endonexin-long; En-L), which does not interact with the β3 tail, acted as a dominant-negative inhibitor of β3-A internalisation. In addition, in the case of the Tyr⁷⁵⁹-Ala substitution mutant α_{11b}β3(Y759A), the anti-β3-MAK-induced endocytosis of the native β3-integrin α_{11b}β3 was drastically reduced and the expression of the long isoform of β3-endonexin (En-L; β3-endonexin-long) resulted in a substantial reduction in the internalisation of wild-type α_{11b}β3. β3-Endonexin therefore couples the β3-A isoforms to a specific receptor recycling path.

It was further recognised that that recycling path affects the mediator reuptake capacity of storage vesicles in human thrombocytes (α-granules) and endothelial cells (Weibel-Palade bodies) and therefore the secretion behaviour thereof. It is known that the secretion of vasoactive substances, for example platelet-derived growth factor (PDGF), endothelial growth factor (EGF), vascular endothelial growth factor (VEGF), various chemokines and cytokines from thrombocyte a-granules, plays a crucial role in acute and chronic vascular diseases. It was furthermore found that it is possible to intervene in those processes by means of β3-endonexin. It was further recognised that it is possible to intervene with β3-endonexin in pro-atherogenic signal paths in human endothelial cells, such as the expression of monocyte chemoattractant protein-1 (MCP-1) and its secretion from human endothelial cells, or in the expression of urokinase-type plasminogen activator receptor (uPAR) on the cell surface, which considerably influences the migration behaviour thereof.

The present invention accordingly relates especially to

(A) a medicament that comprises a compound for influencing β3-integrin-dependent intracellular processes, the compound being characterised in that, for promoting β3-integrin-internalisation-dependent processes and/or for inhibiting the secretion of pro-atherogenic mediators from endothelial cells and for inhibiting the expression of migration-promoting surface receptors on endothelial cells, it has an activating effect on β3-endonexin-short and/or an inhibiting effect on β3-endonexin-long, the compound being selected from

(a) (I) β3-endonexin-short or (II) an expression vector containing DNA coding for β3-endonexin-short;

(b) a ribozyme or a vector coding therefor, characterised in that it can bind specifically to β3-endonexin-long mRNA and cleave the latter, but cannot bind to β3-endonexin-short mRNA, whereby the synthesis of β3-endonexin-long is specifically reduced or inhibited;

(c) an antisense RNA or a vector coding therefor, characterised in that it can bind specifically to β3-endonexin-long mRNA, but not to β3-endonexin-short mRNA, whereby the synthesis of β3-endonexin-long is specifically reduced or inhibited; and

(d) an anti-f53-endonexin-long antibody; and

(B) a medicament that comprises a compound for influencing β3-integrin-dependent intracellular processes, the compound being characterised in that, for inhibiting β3-integrin-internalisation-dependent processes and/or for inhibiting the secretion of pro-atherogenic mediators from thrombocytes, it has an activating effect on β3-endonexin-long and/or an inhibiting effect on β3-endonexin-short, the compound being selected from

(a) (I) β3-endonexin-long or (II) an expression vector containing DNA coding for β3-endonexin;

(b) a peptide or polypeptide containing the NITY motif; and

(c) a compound that inhibits the binding of β3-endonexin-short to the NITY motif of β3-integrin.

The promotion of β3-integrin-internalisation-dependent processes and/or the inhibition of the secretion of pro-atherogenic mediators from endothelial cells and the inhibition of the expression of migration-promoting surface receptors on endothelial cells can be achieved, on the one hand, by inhibition of the expression of β3-endonexin-long (by means of antisense RNAs or ribozymes) or by inhibition of the activity of β3-endonexin-long (for example by means of antibodies) or, on the other hand, for example, by increasing the expression or the activity of β3-endonexin-short (by administration of the protein itself or a vector expressing the protein). The inhibition of β3-integrin-internalisation-dependent processes and/or the inhibition of the secretion of pro-atherogenic mediators from thrombocytes can be effected by inhibition of the activity of β3-endonexin-short by means of a compound that inhibits the binding of β3-endonexin-short to the NITY motif of β3-integrin, for example an antibody directed against the NITY motif. The binding of β3-endonexin-short to the cytoplasmic domain of β3-integrin can also be inhibited by administering a peptide containing the NITY motif (competitive antagonist).

The expressions “β3-endonexin-short” and “an expression vector containing DNA coding for β3-endonexin-short” used herein apply to every form of the protein itself and to the form coded by the DNA, respectively, that is capable of binding to the cytoplasmic domain of β3-integrin β3-A and thereby promoting β3-integrin-internalisation-dependent processes and/or inhibition of the secretion of pro-atherogenic mediators from endothelial cells and inhibition of the expression of migration-promoting surface receptors on endothelial cells, including not only the wild-type form but also forms that, compared with the wild-type form, exhibit changes in the amino acid sequence, such as substitution of one or more amino acids, deletions or additions, without the original biological activity thereby being appreciably changed.

By reducing or inhibiting the expression of β3-endonexin-long, it is likewise possible to promote β3-integrin-internalisation-dependent processes and/or inhibit the secretion of pro-atherogenic mediators from endothelial cells and the expression of migration-promoting surface receptors on endothelial cells. That can be achieved, for example, by ribozymes or antisense RNAs that reduce or eliminate the translation of β3-endonexin-long. Those antisense RNAs and ribozymes are preferably complementary to a coding region of the mRNA.

The person skilled in the art is in a position to produce and use suitable antisense RNAs starting from the published DNA sequences of β3-endonexin-short and β3-endonexin-long (the relevant sequences can be learned from Genbank/EMBL accession number hs371391). Suitable procedures are described, for example, in EB-B1 0 223 399 or EP-A1 0 458. Ribozymes are RNA enzymes and consist of a single RNA strand. They are capable of intra-molecularly cleaving other RNAs, for example the En-L-mRNAs. These ribozymes must in principle have two domains: (1) a catalytic domain and (2) a domain that is complementary to the target RNA and can bind to it, which is the pre-requisite for cleavage of the target RNA. Proceeding from procedures described in the literature, it is now possible specifically to construct ribozymes that cleave a desired RNA at a specific, pre-selected site (see, for example, Tanne et al., in: Antisense Research and Applications, CRC Press, Inc. (1993), 415-426). The above-mentioned ribozymes or antisense RNAs preferably hybridise with En-L-mRNA over a region of at least 15, more preferably at least 25 and most preferably at least 50 bases.

For the insertion and expression of the above-discussed DNA coding for β3-endonexin-short, ribozymes and antisense RNAs it is possible to use a number of expression vectors known to the person skilled in the art. The term “expression vector” refers to a plasmid (pUC18, pBR322, pBlueScript etc.), a virus or another suitable vehicle. The above-mentioned DNA sequences are functionally linked in the expression vector to regulatory elements that permit expression thereof in prokaryotic or eukaryotic host cells. In addition to containing regulatory elements, such vectors contain, for example, a promoter, typically an origin of replication and specific genes that permit the phenotypic selection of a transformed host cell. The regulatory elements for expression in prokaryotes, for example E. coli, include the lac, trp promoter or T7 promoter, and for expression in eukaryotes the AOX1 or GAL1 promoter in yeast and the CMV, SV40, RVSAO promoter, CMV or SV40 enhancer for expression in animal cells. Further examples of suitable promoters are the metallothionein I and the polyhedrin promoter. Suitable expression vectors for E. coli include, for example, pGEMEX, pUC derivatives, pGEX-2T, pET3b and pQE-8, the latter being preferred. Vectors suitable for expression in yeast include pY100 and Ycpad1, for expression in mammalian cells pMSXND, pKCR, pEFBOS, cDM8, pCEV4 and the vectors described in the Examples which follow. Suitable expression vectors also include vectors derived from Baculovirus for expression in insect cells, for example pAcSGHisNT-A.

The DNA sequences described above are preferably inserted into a vector suitable for gene therapy, for example under the control of a tissue-specific promoter, and infiltrated into the cells. In a preferred embodiment, the vector containing the above-described DNA sequences is a virus, for example a retrovirus, adenovirus, adeno-associated virus or vaccinia virus. Examples of suitable retroviruses are MoMuLV, HaMuSV, MuMTV, RSV or GaLV. For the purposes of gene therapy, the DNA sequences according to the invention can also be transported to the target cells in the form of colloidal dispersions. The latter include, for example, liposomes or lipoplexes (Mannino et al., Bio-Techniques 6 (1988), 682). In addition, these vectors can be modified by conventional methods in such a way that they are target-specific. Further suitable vectors and methods for in vitro or in vivo gene therapy are described in the literature and are known to the person skilled in the art; see, for example, also WO 93/04701, WO 92/22635, WO 92/20316, WO 92/19749, WO 92/06180, WO 94/29489 and WO 97/00957. This gene therapy can be employed both for promoting β3-integrin-internalisation-dependent processes and/or inhibiting the secretion of pro-atherogenic mediators from endothelial cells and inhibiting the expression of migration-promoting surface receptors on endothelial cells (for example using a vector containing the gene for β3-endonexin-short or a DNA sequence for an anti-β3-endonexin-long mRNA ribozyme), and for inhibiting β3-integrin-internalisation-dependent processes and/or inhibiting the secretion of pro-atherogenic mediators from thrombocytes (for example using a vector containing a gene for β3-endonexin-long or a DNA sequence coding for a peptide or polypeptide containing the NITY motif).

General methods that are known in the specialist field can be used for the construction of expression vectors that contain the above-mentioned DNA sequences and suitable control sequences. These methods include, for example, in vitro recombination techniques, synthetic processes, and in vivo recombination methods, as are described, for example, in Sambrook et al., supra.

The β3-endonexin-short contained in the medicament according to the invention is preferably produced recombinantly, for example by means of the expression vectors described above. Suitable host cells include, for example, bacteria (for example the E. coli strains HB101, DH1, x1776, JM101, JM109, BL21 and SG13009), yeasts, preferably S. cerevisiae, insect cells, preferably sf9 cells, and animal cells, preferably mammalian cells. Preferred mammalian cells are CHO, VERO, BHK, HeLaCOS, MDCK, 293 and W138 cells. Methods for the transformation of those host cells, for the phenotypic selection of transformants and for the expression of the DNA sequences using the expression vectors described above are known in the specialist field. The person skilled in the art is also familiar with conditions for cultivating transformed or transformed host cells. Suitable purification methods (for example preoperative chromatography, affinity chromatography, for example immunoaffinity chromatography, HPLC etc.) are likewise generally known.

The term “anti-β3-endonexin-long antibody” used herein refers to antibodies or fragments thereof that specifically bind to β3-endonexin-long but not to β3-endonexin-short. These antibodies (and the other antibodies described in connection with the medicament according to the invention) may be monoclonal, polyclonal or synthetic antibodies or fragments thereof. In this context, the term “fragment” means any part of the monoclonal antibody (for example Fab, Fv or “single chain Fv” fragments) that has the same epitope specificity as the complete antibody. The person skilled in the art is familiar with the production of such fragments. The antibodies according to the invention are preferably monoclonal antibodies. The antibodies according to the invention can be produced according to standard procedures, preferably β3-endonexin-long or a synthetic fragment thereof, preferably a fragment having an amino acid sequence that is not present in β3-endonexin-short, serving as the immunogen. The person skilled in the art is likewise familiar with methods of recovering monoclonal antibodies.

In an especially preferred embodiment, the said monoclonal antibody is an antibody originating from an animal (for example the mouse), a humanised antibody or a chimeric antibody or a fragment thereof. Chimeric antibodies resembling human antibodies or humanised antibodies have a reduced potential antigenicity, but their affinity for the target is not reduced. The production of chimeric and humanised antibodies, or of antibodies resembling human antibodies, has been described in detail (see, for example, Queen et al., Proc. Natl. Acad. Sci. USA 86 (1989), 1 0029, and Verhoeyan et al., Science 239 (1988), 1534). Humanised immunoglobulins have variable basic structure regions which largely originate from a human immunoglobulin (referred to as acceptor immunoglobulin) and the complementadtate of the determining regions which largely originate from a non-human immunoglobulin (for example from the mouse) (referred to as donor immunoglobulin). The constant region(s), if present, also originate(s) largely from a human immunoglobulin. In the case of administration to human patients, humanised (and human) antibodies offer a number of advantages over antibodies of mice or other species:

(a) the human immune system ought not to recognise the basic structure or the constant region of the humanised antibody as foreign and therefore the antibody response to such an injected antibody ought to be lower than towards a completely foreign mouse antibody or a partially foreign chimeric antibody;

(b) since the effector region of the humanised antibody is human, it interacts better with other parts of the human immune system, and

(c) injected humanised antibodies have a half-life that is substantially equivalent to that of naturally occurring human antibodies, which makes it possible to administer smaller and less frequent doses in comparison with antibodies of other species.

The expressions “β3-endonexin-long” and “an expression vector containing DNA coding for β3-endonexin-long” used herein apply to every form of the protein itself and to the form coded by the DNA, respectively, that is capable of acting as a dominant-negative inhibitor of β3-A internalisation and thereby inhibiting β3-integrin-internalisation-dependent processes and/or the secretion of pro-atherogenic mediators from thrombocytes, including not only the wild-type form but also forms that, compared with the wild-type form, exhibit changes in the amino acid sequence, such as substitution of one or more amino acids, deletions or additions, without the original biological activity thereby being appreciably changed.

For the insertion and expression of the above-discussed DNA coding for β3-endonexin-long, it is likewise possible to use a number of expression vectors known to the person skilled in the art, the remarks made above concerning the promotion of β3-intregrin-internalisation-dependent processes and/or the inhibition of the secretion of pro-atherogenic mediators from endothelial cells and the inhibition of the expression of migration-promoting surface receptors on endothelial cells applying analogously to these expression vectors. The remarks made above concerning β3-endonexin-short likewise apply analogously to the recombinant production of β3-endonexin-long, suitable expression vectors, host cells, cultivation methods and purification methods.

The expression “peptide or polypeptide containing NITY motif” used herein refers to any kind of peptide or polypeptide that has the NITY motif and that is capable of binding to β3-endonexin-short and thereby preventing the latter from binding to β3-integrin. The NITY motif may be in wild-type form, but also in forms that, compared with the wild-type form, have changes in the amino acid sequence, such as substitution of one or more amino acids, deletions or additions, without the binding affinity to β3-endonexin-short thereby being appreciably changed.

The expression “a compound that inhibits the binding of β3-endonexin-short to the NITY motif of β3-integrin” used herein refers to any kind of compound, for example a synthetic or naturally occurring compound, that is capable of so doing, for example by binding to the NITY motif of β3-integrin, as a result of which the latter is no longer available for binding to β3-endonexin-short. An example of such a compound is an antibody that recognises the NITY motif of β3-integrin. See the above general remarks concerning antibodies. In general, compounds that are capable of inhibiting the binding of β3-endonexin-short to the NITY motif of β3-integrin can be identified and made available by various methods. See the description of the methods mentioned below.

The present invention also allows therapeutic measures to be performed in the case of disorders that are connected with dysregulated β3-integrin-dependent intracellular processes or that can be treated, on the one hand, by promotion of β3-integrin-internalisation-dependent processes and/or by inhibition of pro-atherogenic mediators from endothelial cells and by inhibition of the expression of migration-promoting surface receptors on endothelial cells or, on the other hand, by inhibition of β3-integrin-internalisation-dependent processes and/or by inhibition of the secretion of pro-atherogenic mediators from thrombocytes. The compounds described above can be used for the treatment of conditions that are associated with increased activity of β3-integrin-internalisation-dependent processes and/or with increased secretion of pro-atherogenic mediators from thrombocytes or that can be treated by inhibition of β3-integrin-internalisation-dependent processes and/or by inhibition of the secretion of pro-atherogenic mediators from thrombocytes. Such disorders are preferably syndromes that are accompanied by increased secretion of vasoactive mediators (growth factors, cytokines, coagulation factors and others) from a-granules of thrombocytes or Weibel-Palade bodies of endothelial cells.

On the other hand, the compounds described above can also be used for the treatment of conditions that are associated with reduced activity of β3-integrin-internalisation-dependent processes and/or reduced secretion of pro-atherogenic mediators from endothelial cells and reduced expression of migration-promoting surface receptors on endothelial cells, or that can be treated by promoting β3-integrin-internalisation-dependent processes and/or inhibition of the secretion of pro-atherogenic mediators from endothelial cells and inhibition of the expression of migration-promoting surface receptors on endothelial cells.

These disorders are preferably acute and chronic vascular diseases, such as atherosclerosis and unstable vascular plaques, acute cardiac infarct, stroke, peripheral arterial occlusion disease, chronic venous ulcer of the extremities, restenoses following surgical intervention and others.

The medicaments according to the invention preferably comprise, in addition, a pharmaceutically acceptable carrier. Suitable carriers and the formulation of such medicaments are known to the person skilled in the art. Suitable carriers include, for example, phosphate-buffered saline solutions, water, emulsions, for example oil-in-water emulsions, wetting agents, sterile solutions etc. The medicaments can be administered orally or parenterally. Parenteral administration methods include topical, intra-arterial, intramuscular, subcutaneous, intramedullar, intrathecal, intraventricular, intravenous, intraperitoneal or intranasal administration. The dosage that is suitable will be determined by the physician in charge and depends upon various factors, for example the age, sex and weight of the patient, the nature and stage of the disease, the mode of administration etc.

The present invention further relates to a method by which compounds that inhibit binding of β3-endonexin-short to the NITY motif of β3-integrin can be identified. Such a method comprises incubating β3-endonexin-short with a3-integrin and the compounds to be tested and detecting the binding inhibition of f53-endonexin-short to β3-integrin. It can be advantageous for β3-endonexin-short to be in the form of a peptide or polypeptide that recognises the NITY motif. It can also be advantageous for β3-integrin to be in the form of a peptide or polypeptide that comprises the NITY motif. It can further be advantageous for there to be a combination of the two alternatives. It can furthermore be advantageous for β3-endonexin-short or the peptide or polypeptide thereof that recognises the NITY motif and/or for β3-integrin or the peptide or polypeptide comprising the NITY motif to be in the form of expression vectors that encode them.

An especially advantageous method is one in which β3-endonexin is immobilised on avidin microtitre plates after biotinylation and the NITY motif, in the form of a 10-mer peptide coupled to fluorescein, is added together with a compound to be tested. The binding inhibition of the compound being tested is ascertained by determining the fluorescence.

Another especially advantageous method is one in which β3-endonexin-short, fused to the green fluorescent protein (GFP), is immobilised on microtitre plates as described above, and the above-mentioned 10-mer peptide is added together with the compound to be tested. The binding inhibition of the compound being tested is ascertained by determining the resonance energy transfer between fluorescein and GFP.

Yet another especially advantageous method is one in which cells, for example CHO cells, that express GPIIb/IIIa and β3-endonexin-short are used. After the addition of a compound to be tested, the binding inhibition of the compound by means of GRIIb/IIIa internalisation is determined by measurement by way of antibody binding and FACS.

### BRIEF DESCRIPTION OF THE FIGURES

FIG. 1: Diagram of the Chimeric Receptors Used

The amino acid sequences of the cytoplasmic domains of β1-A, wild-type β3-B, alternatively spliced forms of β3 and mutant β3-A are shown. Intracellular domains of β3-integrins (wild-type and mutants) were expressed as chimeric single-chained receptors that had been fused to heterologous extracellular domains and transmembrane domains corresponding to the CH2 and CH3 domains of human IgG, and of the CD7 antigen, respectively. Underlining indicates changed amino acid sequences.

FIG. 2A: Double Immunofluorescence Micrograph of CHO Cells Transiently Transfected with β3 Isoform Fusion Proteins: the Surface Expression of Ig-β3-A is Considerably Reduced

CHO cells were plated onto cover glasses and left to grow to subconfluence. The cells were then transfected with aliquots of plasmid DNAs coding for various Ig-β3-integrin chimeras (β3-A, β3-B, β3-C, β3-A) or Ig control constructs. After 24 hours, the cells were fixed and the cell surface expression of the fusion proteins was detected by staining with a TxR-conjugated mouse anti-IgG, antibody (red immunofluorescence). The cells were then permeabilised with 0.02% Triton X-100™ and the intracellular expression of the chimeras was detected by staining with a FITC-conjugated mouse anti-IgG, antibody (green fluorescence). It will be noted that all of the chimeras are expressed diffusely (green) within the cells. Significantly, all of the chimeras with the exception of Ig-β3 can be detected on the cell surface (red).

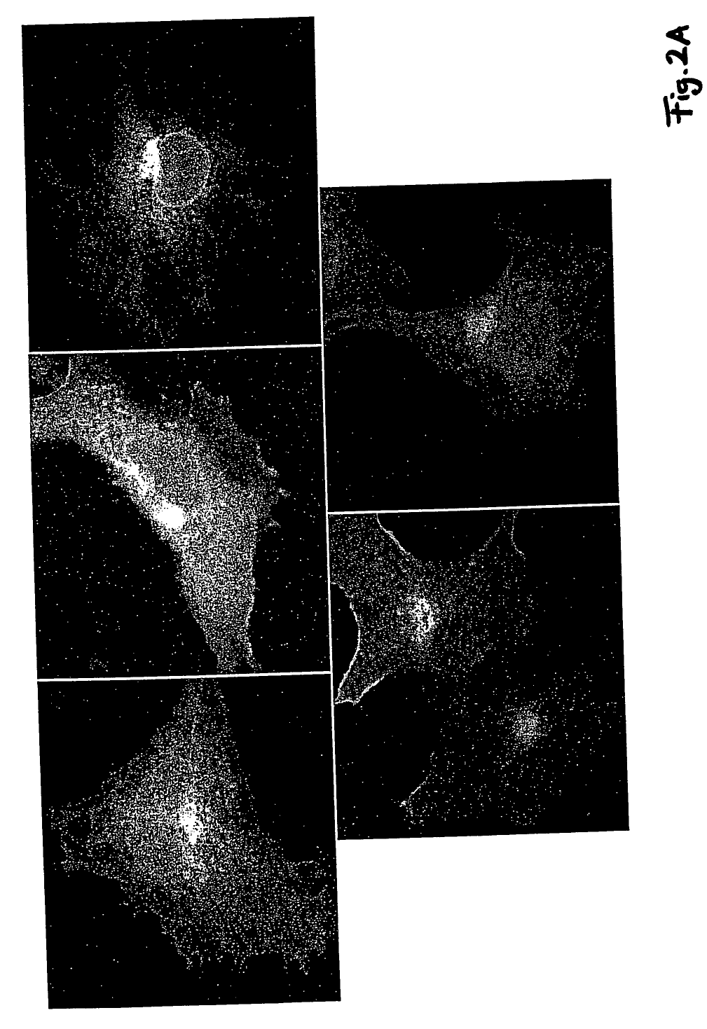

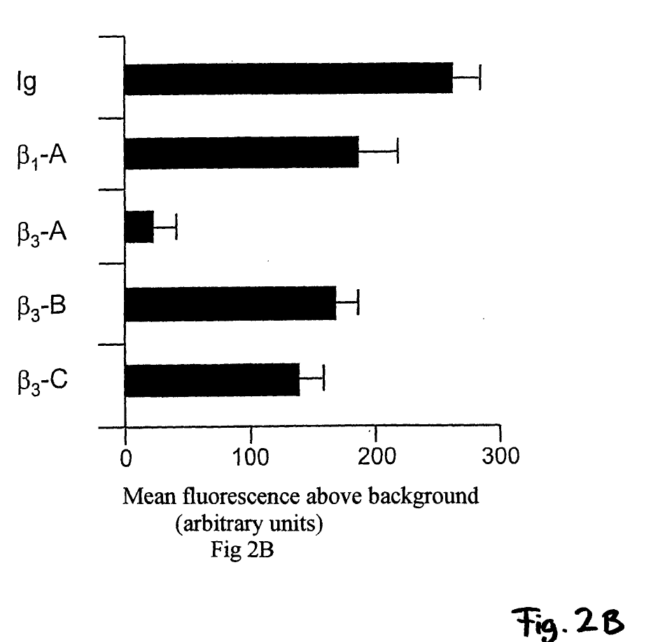

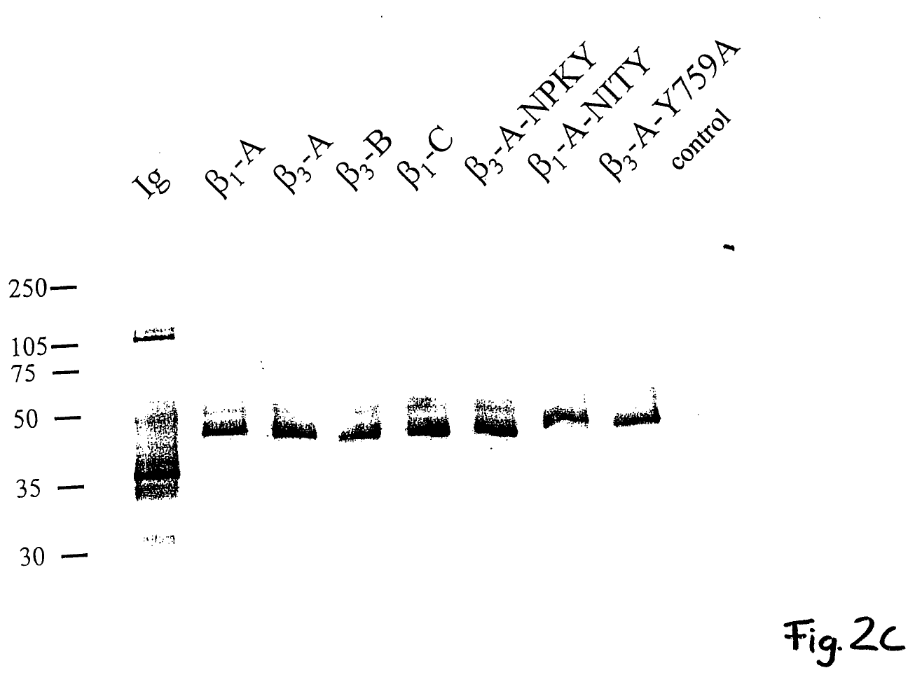

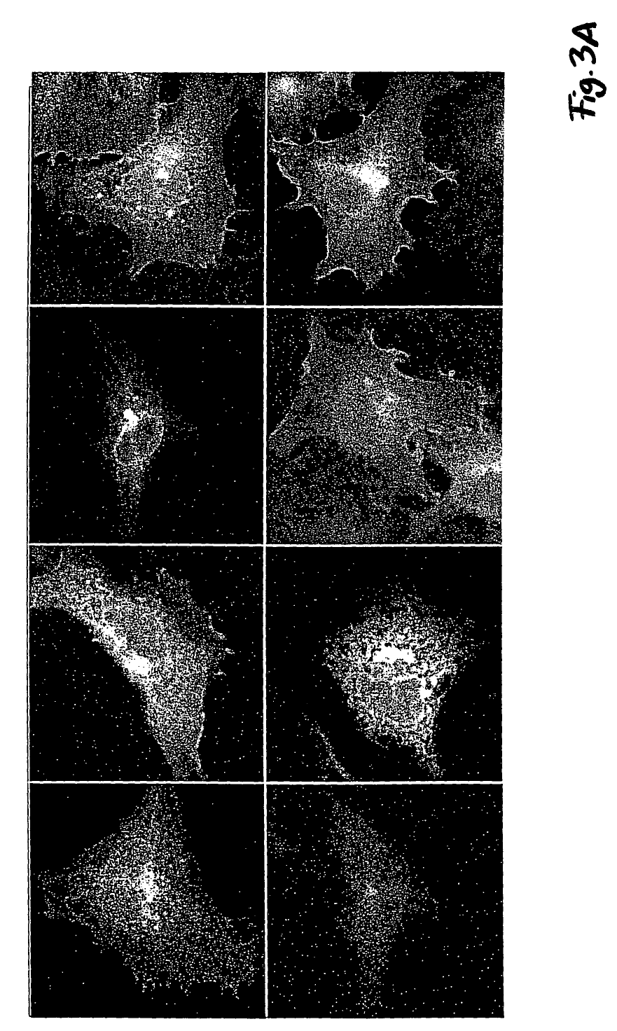

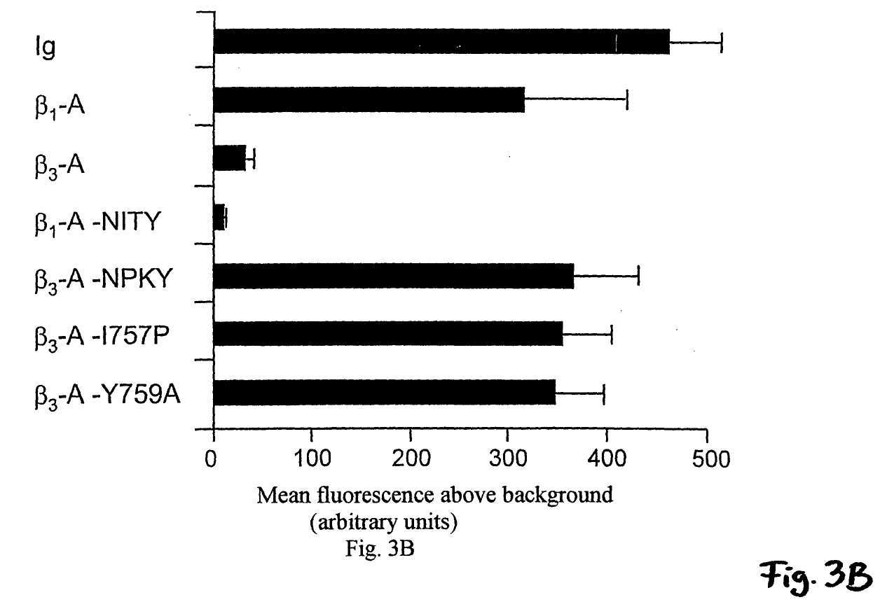

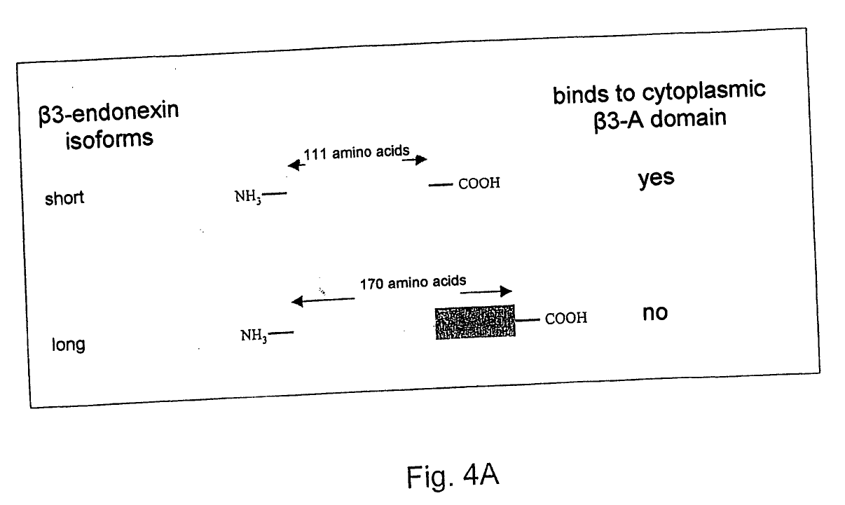

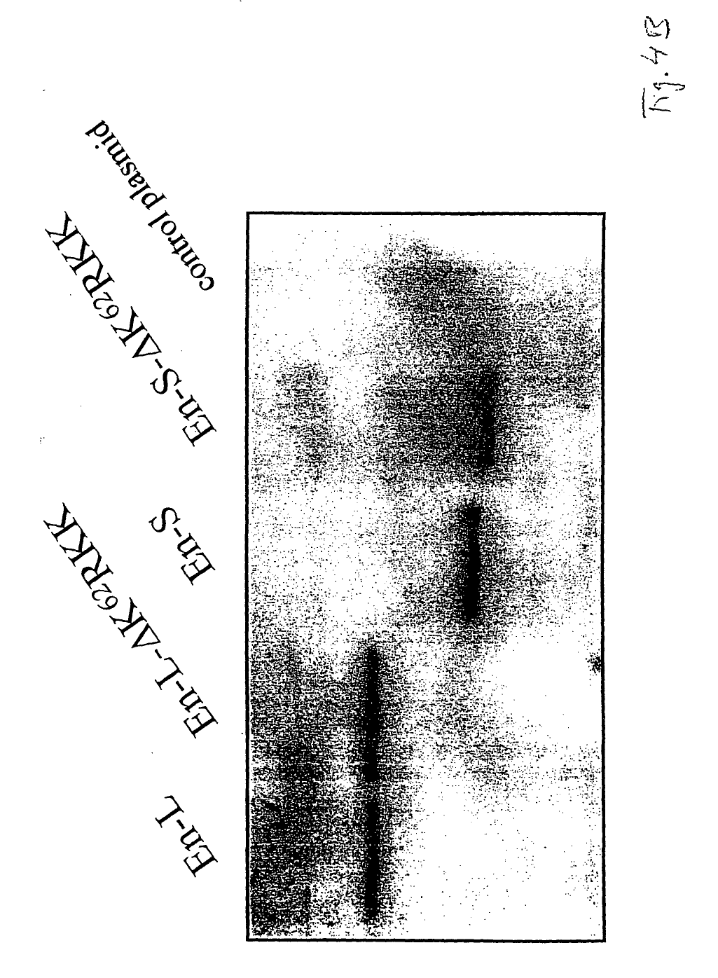

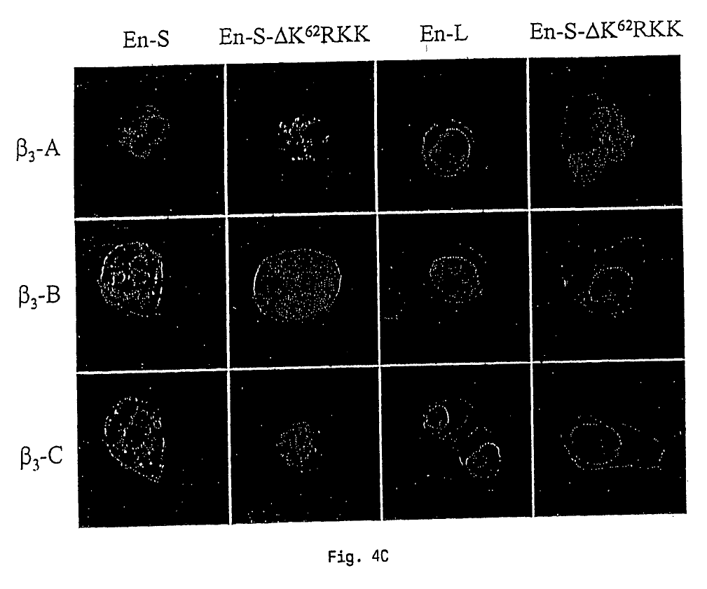

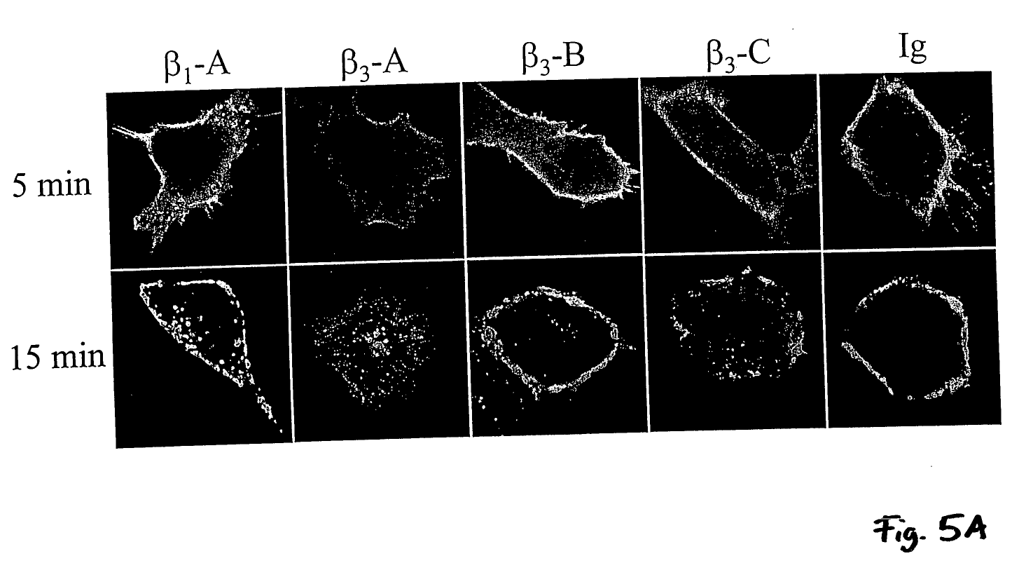

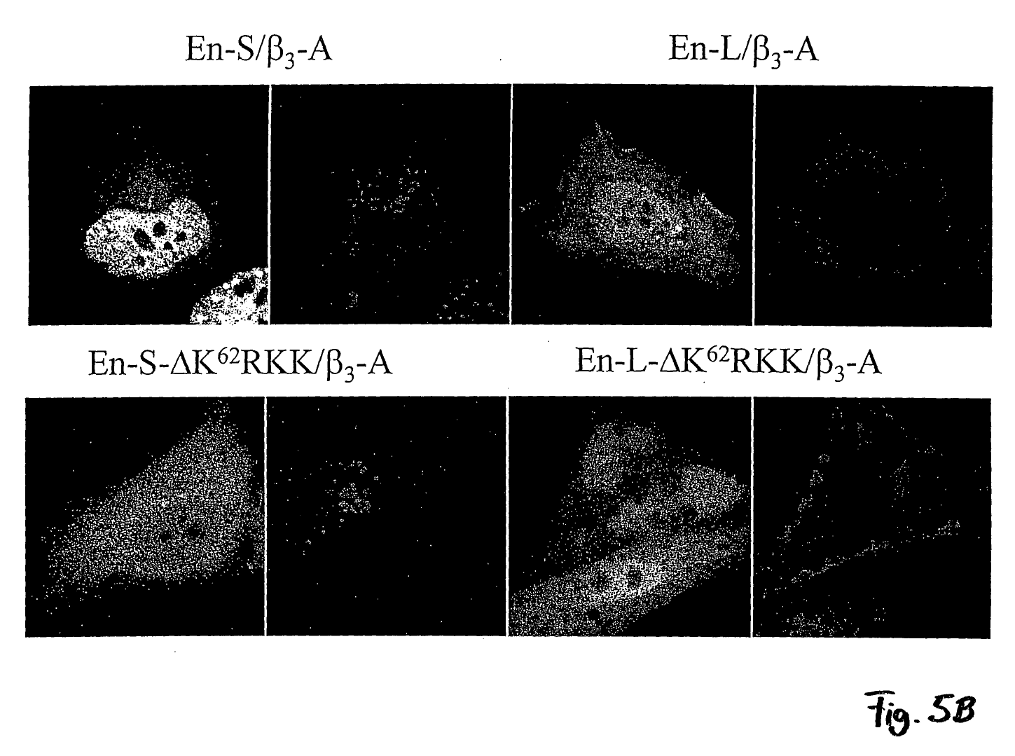

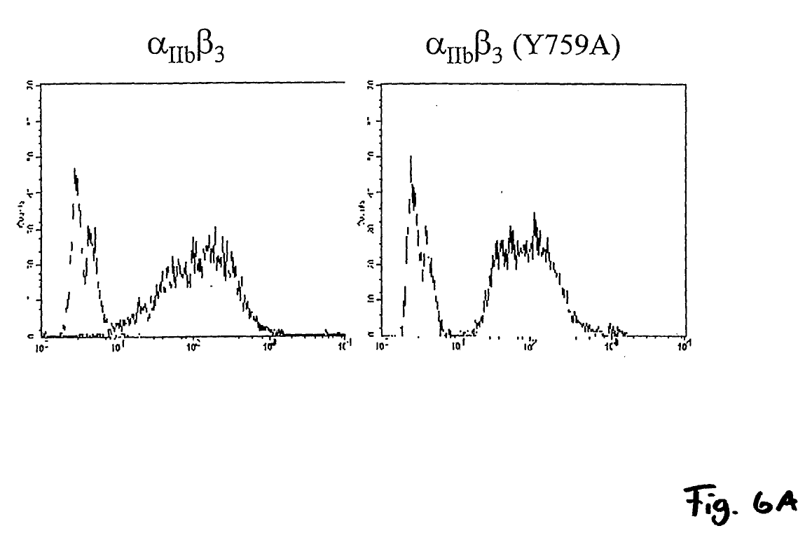

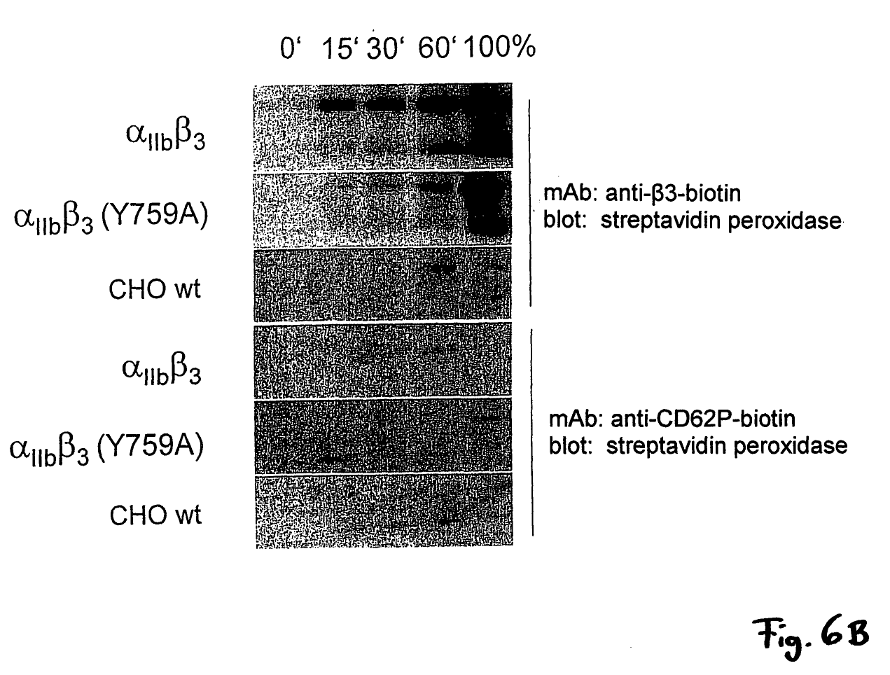

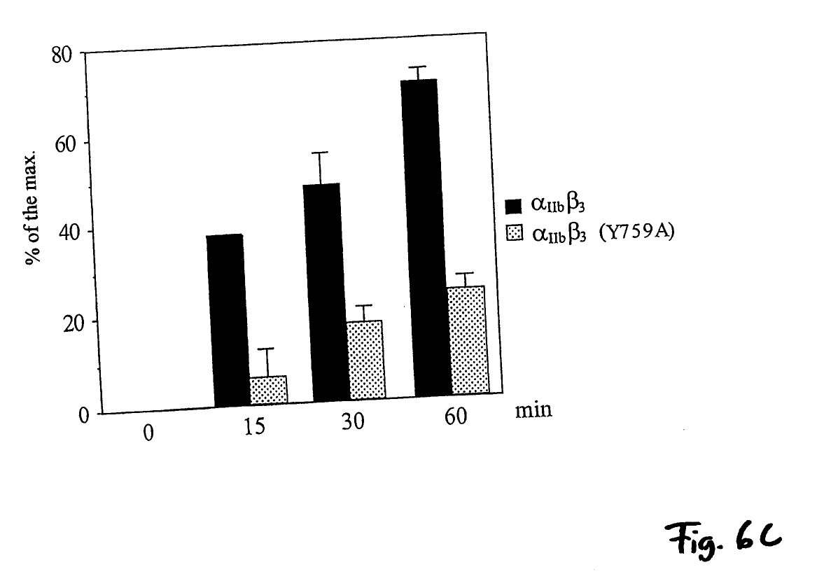

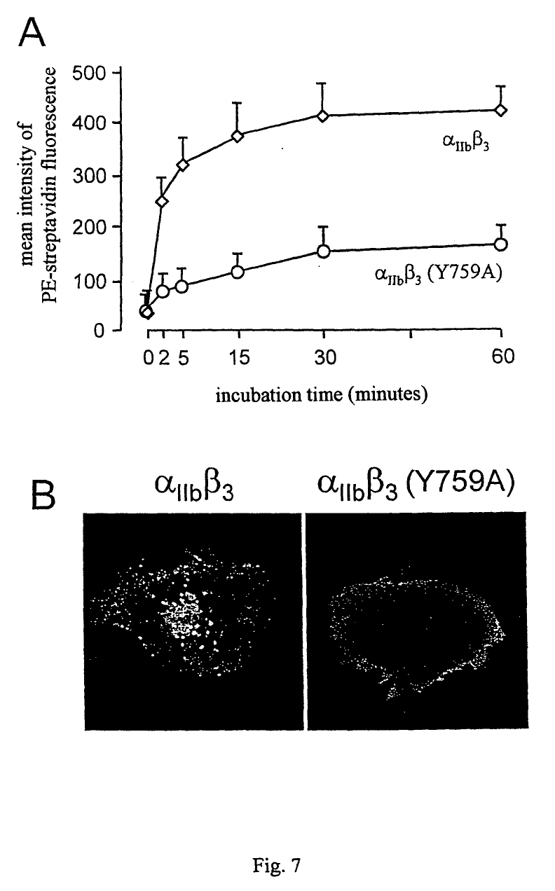

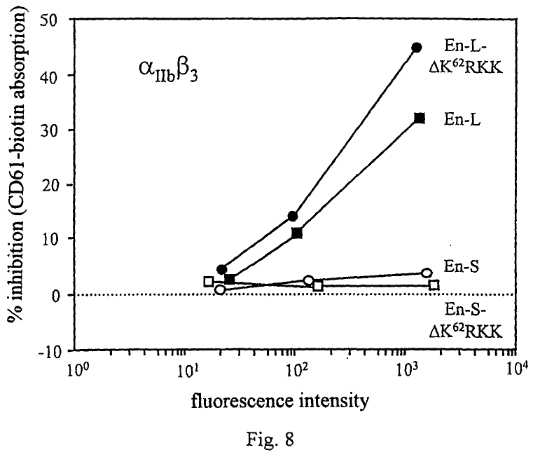

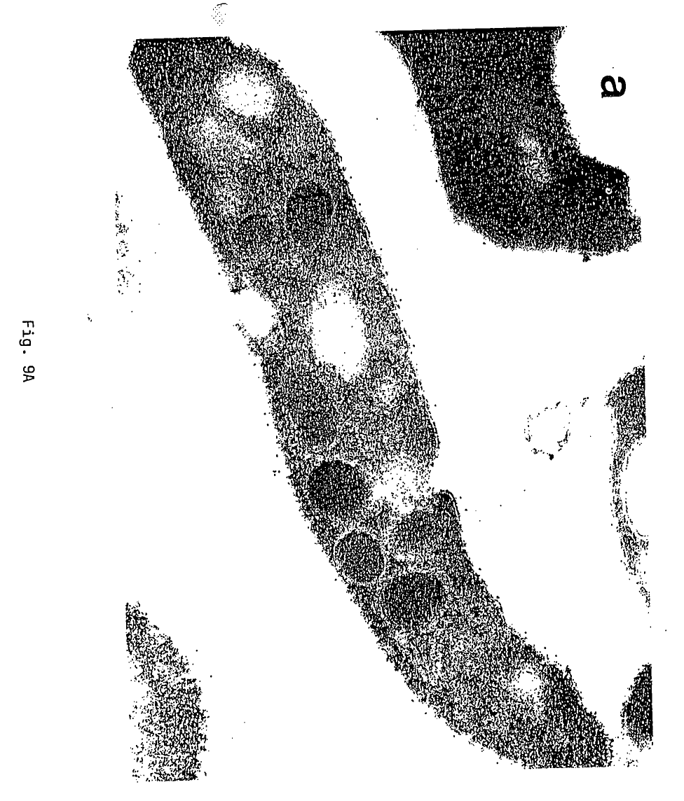

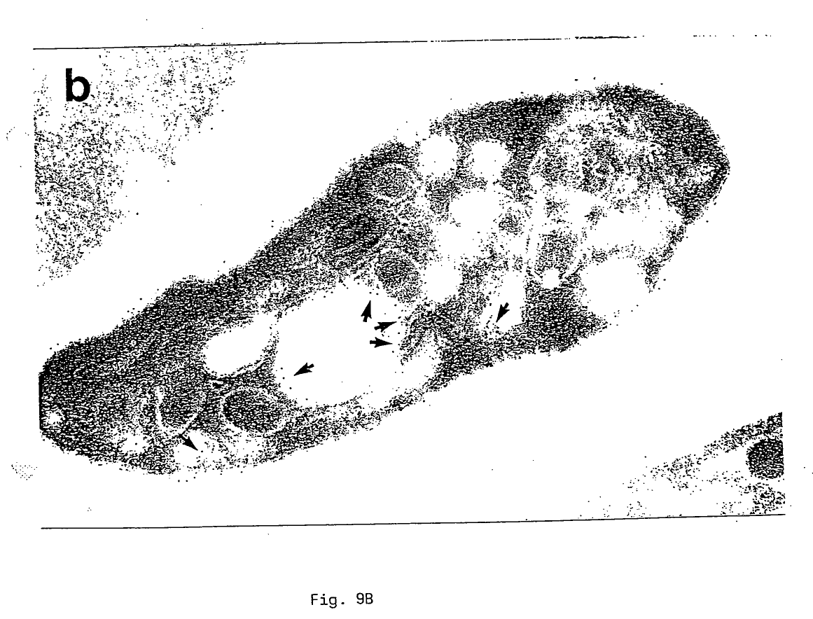

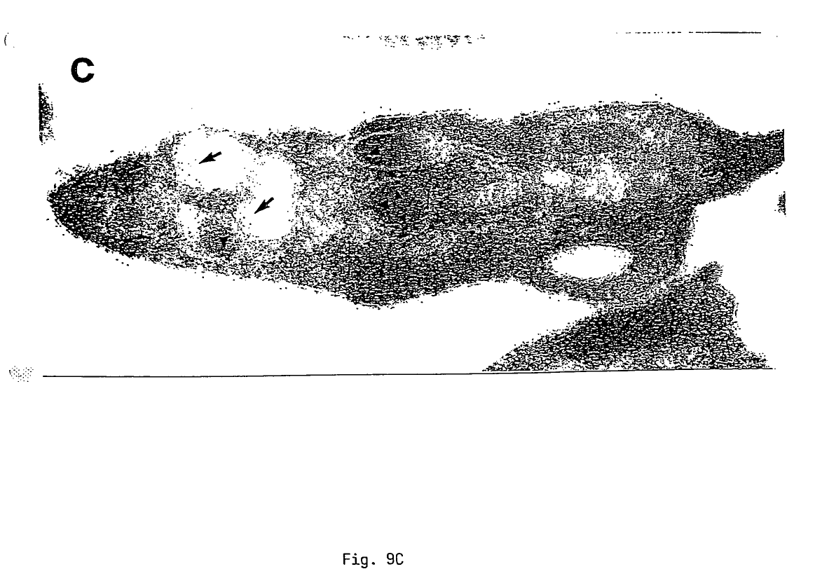

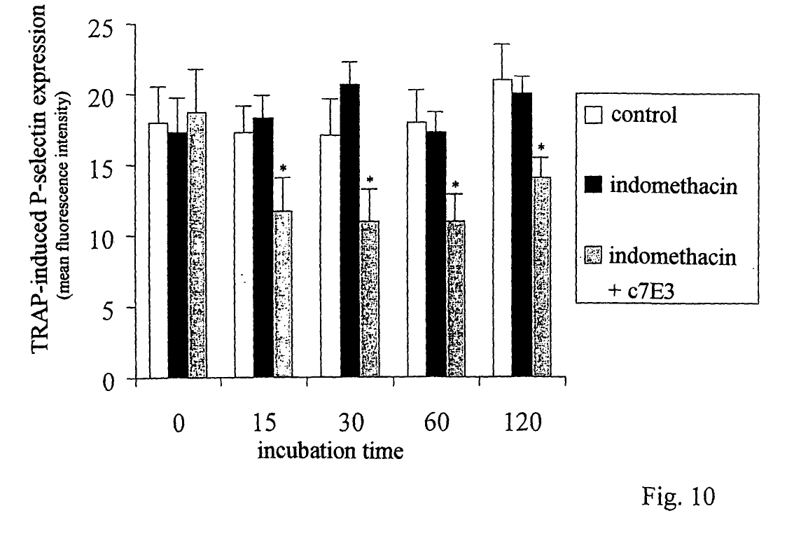

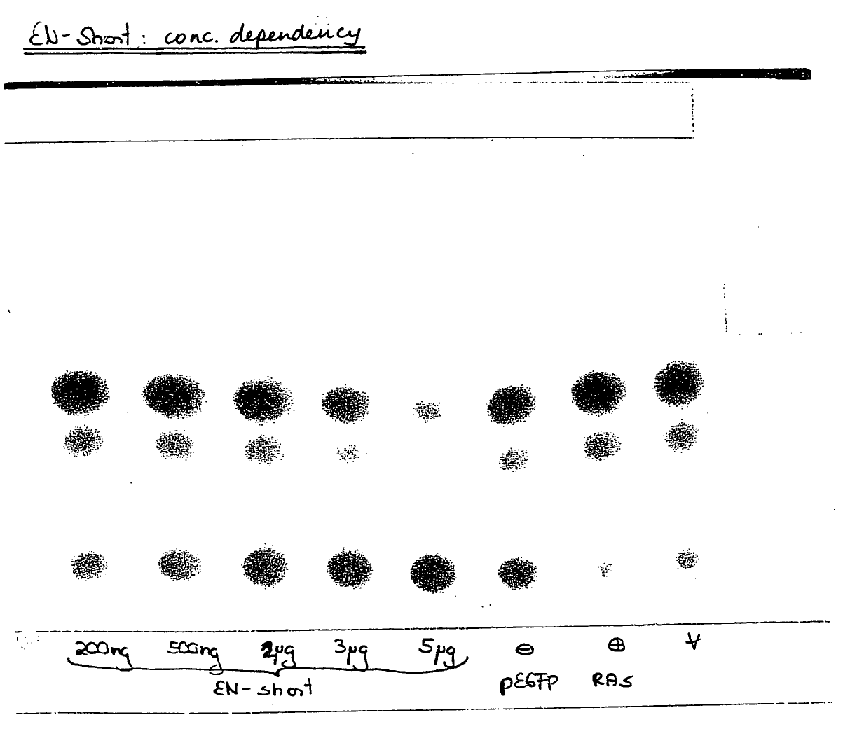

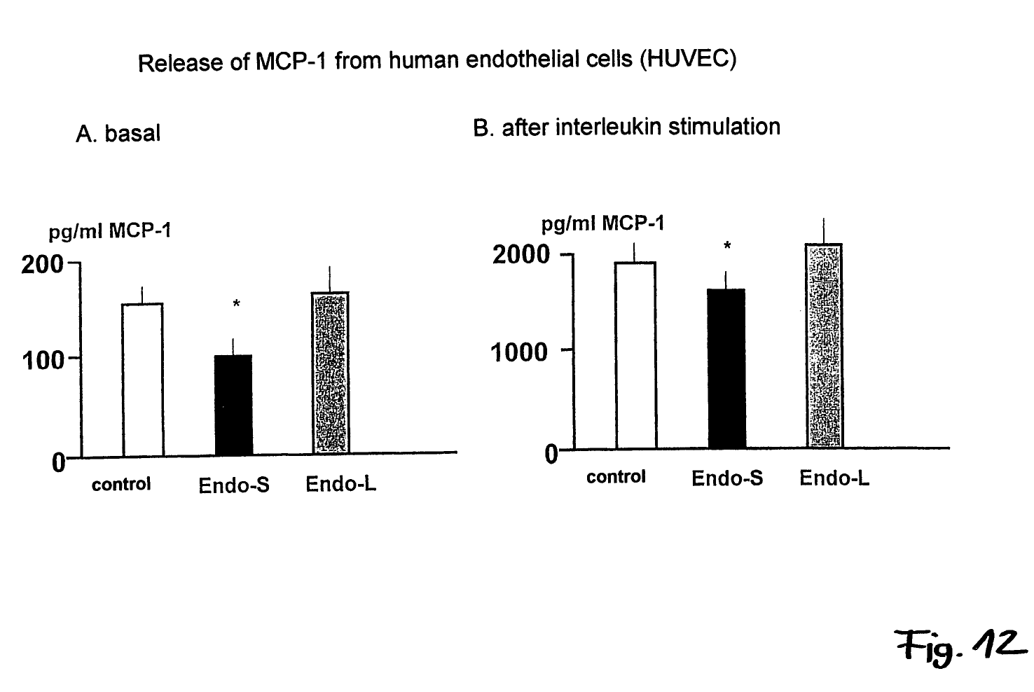

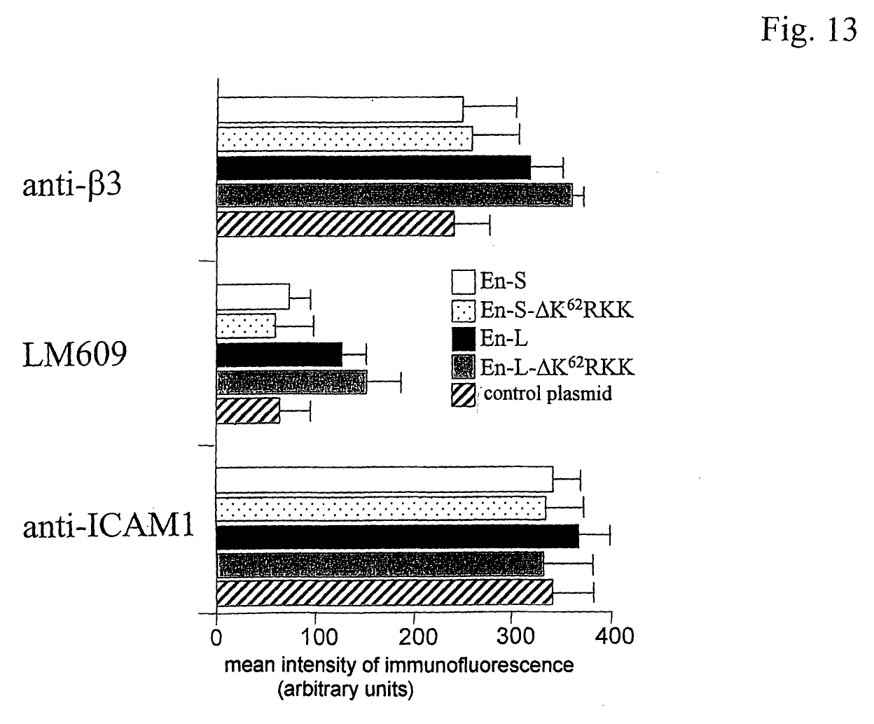

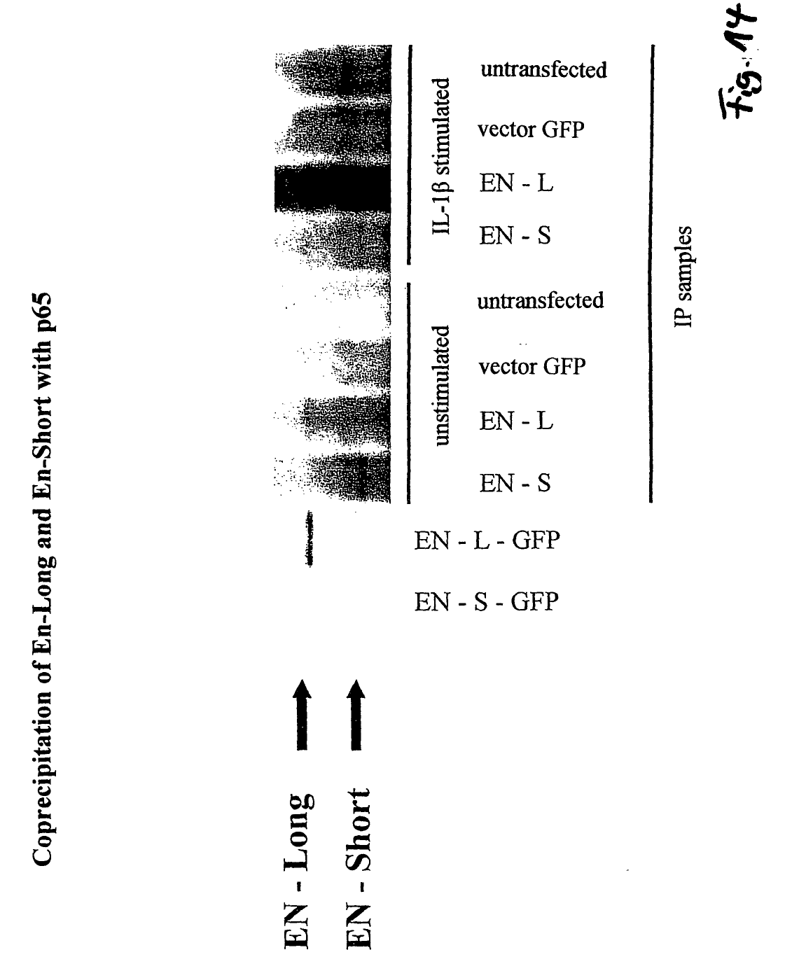

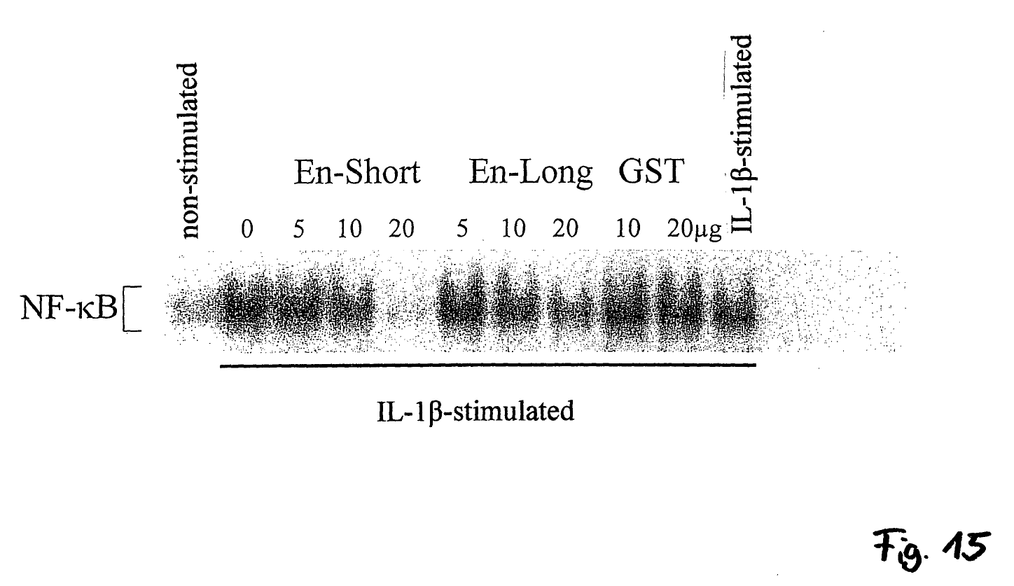

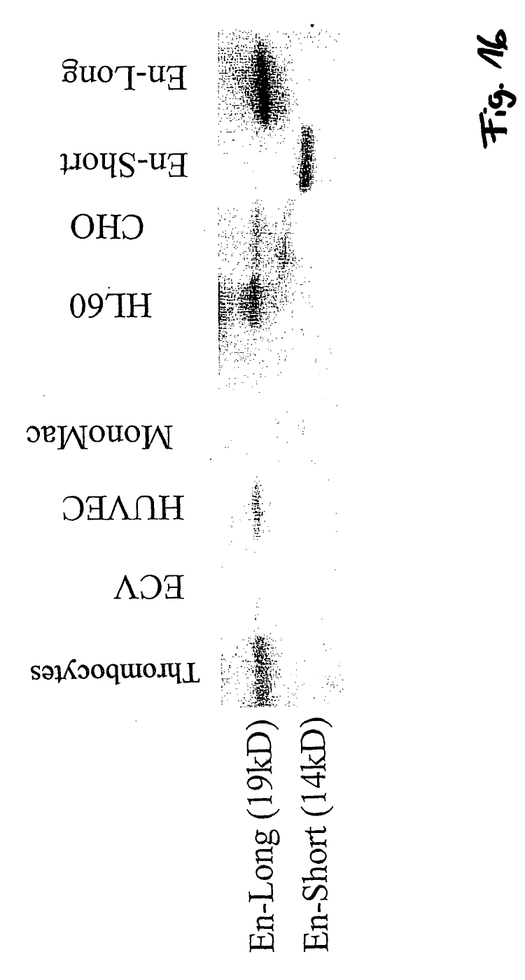

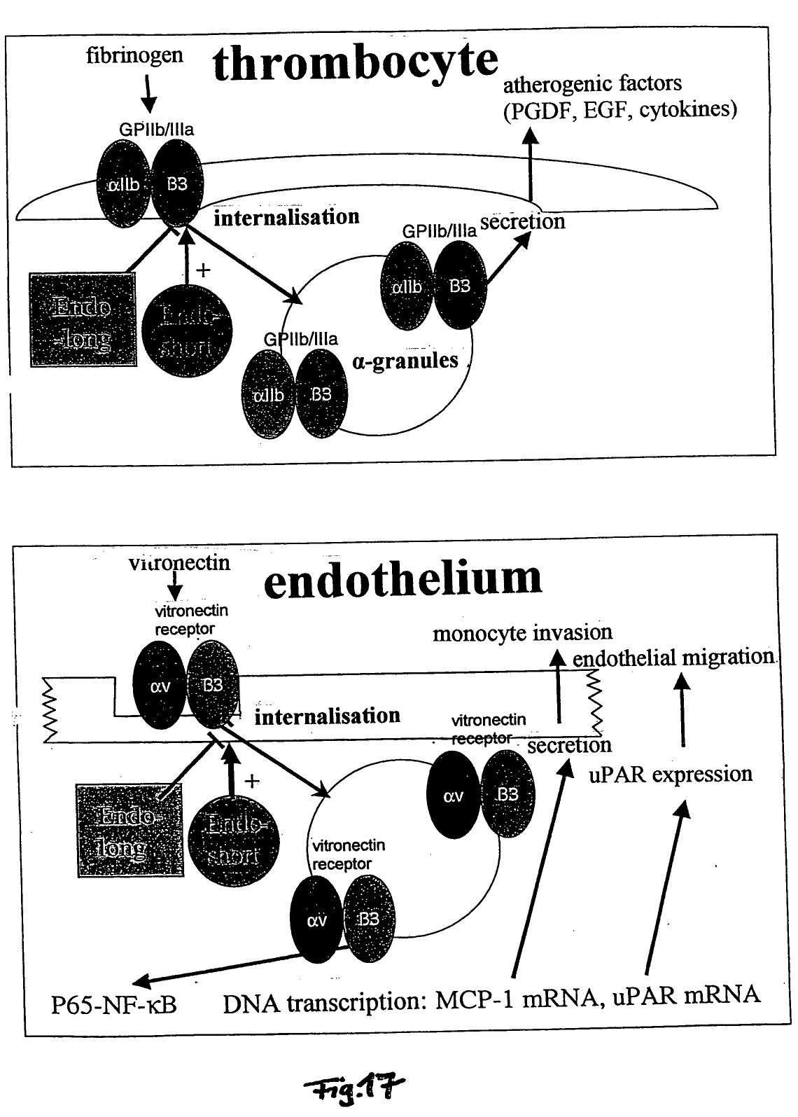

FIG. 2B: Flow Cytometry Analysis of the Cell Surface Expression of β3 Isoform Chimeras in CHO Cells

Subconfluent monolayers of CHO cells were transiently transfected with chimeric constructs of the β3 isoforms (Ig-β3-A, Ig-β3-B, Ig-fb3-C). The Ig-131-A chimeras or the tailless Ig control construct were used as controls. The cells were fixed with 2% formaldehyde and stained with a TxR-conjugated mouse anti-IgG, antibody. The cells were then washed and resuspended in 0.02% Triton X-10d™ and PBS containing saturation concentrations of FITC-conjugated mouse anti-IgG₁ antibody for detection of the intracellular expression of the corresponding chimeras. Samples were then analysed by flow cytometry. The mean values of the results of three experiments performed independently are shown. Background immunofluorescence levels were defined by means of “mock” transfectants (vector DNA).

FIG. 2C: Expression of β1 and β3 Isoform Chimeras

Subconfluent layers of COS cells were transiently transfected with the chimeric constructs indicated. After 24 hours, the cells were lysed and the Ig fusion proteins were precipitated on protein A-sepharose. Immunoblot analysis by means of an anti-Ig antibody was then carried out.

FIG. 3A: Two-colour Immunofluorescence Micrograph of CHO Cells Transiently Expressing Fusion Proteins of β-β3-integrin-NITY Motif-mutants or Variants: Chimeras Carrying the Cytoplasmic NITY Motif are Localised within the Cell

CHO cells were plated out onto cover glasses pre-coated with vitronectin and were transiently transfected with the β3-integrin mutants β1-A-NITY, β3-A-NPKY, β3-A-1757P, β3-A-Y759A, the β3-A and β1-A isoforms or the tailless Ig control vector. After 24 hours, the cells were fixed and stained as described in the legend to FIG. 2. The red immunofluorescence indicates surface expression and the green indicates intracellular expression of the chimeric constructs. It will be noted that all of the chimeras are expressed equally and diffusely within the cell (green) and that point mutations or deletions of the NITY motif lead to increased cell surface expression.

FIG. 3B: Quantification of the Cell Surface Expression of NITY Motif Mutants

CHO cells were transiently transfected with chimeric constructs of the β3-NITY mutants. The cells were stained and the cell surface expression of the chimeras was analysed as described in the legend to FIG. 2. The mean values of the results of three experiments performed independently are shown.

FIG. 4A: Diagram of the β3-endonexin Isoforms

FIG. 4B: Western Blot Analysis of GFP-endonexin Fusion Proteins in CHO Cells

Aliquots of entire lysates of CHO cells that had been transfected with the constructs indicated were separated by SDS-PAGE, and an immunoblot analysis was carried out by means of an anti-GFP antibody.

FIG. 4C: Cell Surface Expression of β3-isoform-Ig Fusion Proteins (Red) in CHO Cells Co-transfected with GFP Fusion Proteins of β3-endonexin Isoforms (Green): GFP-En-L and GFP-En-L-ΔK⁶²RKK Mediate Specifically the Cell Surface Expression of an Ig-β3-A Chimera but Not That of Ig-β3-B or Ig-β3-C Chimeras

CHO cells were transiently co-transfected with β3-integrin and GFP/β3-endonexin isoforms. After 24 hours, the cells were fixed and stained with a TxR-conjugated mouse anti-IgG₁ antibody (red immunofluorescence) with regard to the cell surface expression of β3 chimeras. It will be noted that Ig-β3-A is expressed on the cell surface of cells that have been co-transfected with the long form of β3-endonexin (top row, third image from the left).

FIG. 4D: Quantification of the Cell Surface Expression of Chimeras of the Cytoplasmic Domain of β3-integrin in CHO Cells Co-transfected with β3-endonexin Isoforms

CHO cells were transiently transfected with Ig-β3-A, Ig-β1-A, Ig-β1-NITY or Ig-β3-NPKY expression constructs together with GFP/β3-endonexin isoforms. After 24 hours, the cells were fixed and stained with TxR-conjugated mouse anti-IgG₁ (red immunofluorescence) with regard to the cell surface expression of the fusion proteins. 10,000 green (GFP)-positive cells were assessed. The mean values of the results of three experiments performed independently are shown.

FIG. 5A: An Exogenous Anti-Ig Antibody is Rapidly Internalised by Cells Expressing Ig-β3-A

CHO cells were plated out onto cover glasses pre-coated with vitronectin and were transiently transfected with Ig-β3-A, Ig-β3-B, Ig-β3-C, Ig-β1-A or the tailless Ig control constructs. After 24 hours, 5 μl/ml of TxR-conjugated anti-IgG₁-MAK were added to the culture medium and incubation was carried out for 15 minutes. Internalisation was stopped by the addition of ice-cold fixing agent to the cells at the intervals indicated. The cells were then assessed by means of immunofluorescence microscopy. It will be noted that, in contrast to the other chimeras, β3-A-transfected cells do not exhibit any appreciable cell surface staining. The green immunofluorescence, however, accumulates in intracellular vesicular compartments within minutes.

FIG. 5B: β3-endonexin-L Constructs Block the Constitutive Internalisation of Ig-β3-A

CHO cells were plated out onto cover glasses pre-coated with vitronectin and were transiently co-transfected with the β3-integrin-β3-A and β3-endonexin isoforms. After 24 hours, 5 μl/ml of TxR-conjugated anti-IgG₁-MAK were added to the culture medium and incubation was carried out for 15 minutes. Internalisation was stopped by the addition of ice-cold fixing agent to the cells at the intervals indicated. The cells green-fluorescing cells were then assessed by means of two-colour immunofluorescence microscopy. It will be noted that, in cells transfected with En-L constructs, the internalisation of β3-A is considerably reduced.

FIG. 6: Measurement of the Endocytosis of Native β3-integrin: Biochemical Assessment

(A) Flow cytometry analysis of stable cell lines expressing a_{IIb} with wild-type β3 or the β3 mutant Y759A. Fluorescence histograms with negative control antibody (anti-CD62, left) or anti-β3 (mAb 15) (right) conjugated with fluorescein isothiocyanate are shown.

(B) CHO cells were pre-incubated on ice for 15 minutes with biotin-labelled anti-β3-MAK and then incubated at 37° C. for the periods indicated. After incubation with the reducing agent, the cells were lysed and the proteins were separated by SDS-PAGE and detected by means of immunoblotting using peroxidase-coupled streptavidin. For the semi-quantitative assessment of the fraction of biotin-labelled MAKs that were protected by the reducing agent, aliquots of parallel samples were taken after cold incubation and, to make it easier to estimate the internalised material as a fraction of the total input, were loaded as standardisation controls (lanes on the right, 100, 50, 10%, α_{IIb}β3, and 80, 40, 20%, α_{IIb}β3 (Y759A)). All samples were taken as duplicates.

(C) Corresponding results of density scanning of the antibody bands (OD=optical density, arbitrary units).

FIG. 7: Measurement of the Endocytosis of Native β3-integrin: Immunofluorescence

(A) Quantitative assessment of the internalisation of biotin-labelled anti-β3-MAK in CHO cells expressing wild-type α_{IIb}β3 and α_{IIb}β3 (Y759A). The cells were incubated on ice for 15 minutes with a biotin-labelled anti-β3-MAK (5 μg/ml), then diluted with culture medium and incubated at 37° C. for the periods indicated. This was followed by washing steps with Dulbecco's modified PBS and three successive incubations with a solution containing 50 mM 2-mercaptoethanesulfonic acid (Sigma), 10 mM NaCl, 1 mM EDTA, 50 mM Tris and 0.2% bovine serum albumin, pH value 8.6. The cells were then fixed with 2% formaldehyde, permeabilised with 0.2% Triton X-100™, stained with FITC-conjugated streptavidin and analysed by means of flow cytometry.

(B) Qualitative comparison of the absorption of wild-type α_{IIb}β3 and α_{IIb}β3 (Y759A); the cells were treated as described above. A typical cell of the 15 minute sample is shown. The analyses were carried out by means of a confocal laser scanning microscope (Leica).

FIG. 8: Dosage-dependent Analysis of the Endocytosis of Native Wild-type β3-integrin in Cells Transiently Transfected with β3-endonexin Isoforms

CHO cells expressing wild-type α_{IIb}β3 were transiently transfected with GFP/β3-endonexin constructs or GFP control vectors for 24 hours. The cells were pre-incubated with biotin-labelled anti-β3 at 4° C. for 15 minutes and then incubated at 37° C. for a further 15 minutes. After reduction with 2-mercaptoethanesulfonic acid, the cells were stained with PE-streptavidin, and approximately 10⁸ GFP-positive cells were examined by means of flow cytometry. The mean intensity of PE-streptavidin was used as an index of β3-integrin endocytosis and was determined at various GFP mean expression levels relative to the GFP control vector. This was done by analysing the red fluorescence within various PMT fluorescence gates for various levels of the GFP fluorescence (green). The results of one experiment are shown; comparable results were observed in three independent experiments.

FIG. 9: Time-dependent β3-integrin Internalisation in Storage Vesicles (α-granules) of Thrombocytes from Patients with Coronary Heart Disease

Electron micrographs of washed blood platelets (thrombocytes) of patients treated for acute coronary syndrome. The thrombocytes were incubated at room temperature with agonistic antibodies to the β3-integrin glycoprotein IIb/IIIa (7E3 antibody), then with gold-labelled anti-mouse Fab fragments for 10 minutes (A), 20 minutes (B) or 40 minutes (C). After 10 minutes, the immuno-labelled 7E3 antibody (arrows) was located exclusively on the thrombocyte surface. After 20 minutes, the surface labelling was reduced; labelled 7E3 was increasingly located in the surface-connected membrane system (“SCS”) of the α-granules. After 40 minutes, most of the GP IIb/IIIa-indicating 7E3 antibody is demarcated in the α-granules.

FIG. 10: Integrin Internalisation in α-granules Causes Reduced α-degranulation, Measured as TRAP-induced P-selectin Surface Expression on Human Thrombocytes

Effect of the glycoprotein IIb/IIIa antagonist Abciximab on the P-selectin surface expression in TRAP (thrombin receptor activating peptide)-activated thrombocytes. After intravenous administration of Abciximab (Eli-Lilly, Germany), thrombocyte-rich blood plasma was taken at the times indicated from patients being treated for acute coronary syndrome, and the TRAP (thrombin receptor activating peptide; 25 μM)-induced P-selectin expression was determined. The antibody CD62P used recognises that surface selectin which is expressed there after degranulation. The isolated thrombocyte-rich plasma was then incubated in vitro with indomethacin (60 μM) alone or indomethacin and 7E3 antibodies (50 μg/ml). The P-selectin surface expression was then determined by flow cytometry.

* indicates significant change in comparison with the initial value (p<0.05).

FIG. 11: β3-Endonexin-short Down-regulates the uPAR Promoter

CHO cells were transiently transfected with the CAT reporter gene which was under the control of the urokinase-type plasminogen activator receptor (uPAR) promoter. In addition, various amounts of β3-endonexin-short plasmid (200 ng to 5 μg; lane 1-5), the empty expression vector (pEGFP, lane 6) or empty liposomes (lane 8) were co-transfected. Cell extracts that had been normalised with regard to luciferase activity were incubated with ¹⁴C-chloramphenicol, extracted with ethyl acetate and subjected to thin-layer chromatography. The conversion of ¹⁴C-chloramphenicol to acetylated derivatives was carried out by means of a phosphorimager. The results are shown in FIG. 11.

FIG. 12: Effect of the Expression of β3-endonexin-short and β3-endonexin-long on the Secretion of MCP-1 from Endothelial Cells

Cultivated HUVEC cells were transfected either with a plasmid construct coding for β3-endonexin-short or β3-endonexin-long as described in Examples 5 and 6 or with an empty control vector. 24 hours later, the cells were incubated for 120 minutes either with vehicle or with 100 pg/ml of interleukin-1β. The supernatant was then removed by suction and the concentrations of monocyte chemotactic protein 1 (MCP-1) were determined by means of ELISA. The bars represent results of four independent experiments, giving the average standard deviation.

FIG. 13: Over-expression of Endonexin-long Increases the Density of Vitronectin Receptors (VNR) on Human Endothelial Cells (Flow Cytometry Analysis)

Freshly isolated human umbilical endothelial cells (HUVEC cells) were transfected with plasmids that coded for both β3-endonexins or their nuclear import mutants. The cells were harvested, fixed as described and stained with antibodies. The “anti-β3” antibody directed against β3-integrins and the LM 609 antibody directed against the vitronectin receptor (VNR) complex were used for this. An antibody directed against the adhesion receptor ICAM-1 was used as a control. Mean values of three independent tests in each case are shown. The results of both antibodies directed against the VNR concur in showing that the density of the VNR markedly increased after over-expression of En-L.

FIG. 14: Immunoprecipitations of HUVEC Cells Show a Co-precipitation of β3-endonexin-short and β3-endonexin-long with the p65 Sub-unit of NF-kB.

HUVEC were transfected with recombinant En-S or En-L. 24 hours later, extracts of those cells, which were examined under basal conditions or were stimulated with interleukin-1β, were lysed and purified as described. Immunoprecipitation was then carried out with antibodies to NF-kB (biotin-conjugated mouse anti-p65 mAb, Roche Biochemicals, Mannheim). Those precipitates were applied to SDS polyacrylamide gels, transferred to nitrocellulose and blotted with the described antibodies to β3-endonexin. The results show that, especially after cytokine-stimulation, En-S and En-L bind directly to p65 of NF-kB and interact therewith.

FIG. 15: Gel Shift Assays for Measuring NF-kB Activity Show a Neutralisation of the Activatability by Various Cytokines in the Presence of Recombinant β3-endonexin-short and β3-endonexin-long:

Nuclear extracts were prepared from IL-1β-stimulated HUVEC and analysed as described. NF-kB binding is neutralised by over-expression of En-S in HUVEC and markedly reduced by En-L. The protein GST used for purification served as a negative control. The results show that the NF-kB binding activity decreases markedly in the presence of increasing doses of En-S and En-L. The functional significance of the interaction of En-S and En-L with p65 thereby also becomes clear.

FIG. 16: β3-Endonexin-long, Not β3-endonexin-short, is the Dominant Protein in Human Thrombocytes and Endothelial Cells:

The blot shows extracts of human thrombocytes and endothelial cells that had been blotted with the specific antibody to endonexin. The immortalised endothelial cell line ECV and primary-cultivated umbilical endothelial cells (HUVEC) were used. Further cell lines (“L-60”, “CHO”) and purified recombinant proteins (lanes on the right) were used as controls. It is possible to see a clear band at the level of β3-endonexin-long, whereas no endogenous β3-endonexin-short can be distinguished. These findings demonstrate that En-L is the dominant protein in human thrombocytes and endothelial cells.

FIG. 17: Diagram of the Functional Relationship between β3-endonexin-short, β3-endonexin-long and Further Intracellular and Extracellular Components in the Case of a Thrombocyte (FIG. 17A) and in an Endothelial Cell (FIG. 17B).

The following Examples illustrate the invention.

### EXAMPLE 1

### General Procedures

(A) Antibodies and Reagents

MAK Ab-15 is directed against the extracellular domain of β3 and was conjugated with biotin according to standard procedures. Anti-IgG₁ and polyclonal rabbit anti-GFP antibodies were obtained as fluorochrome conjugates (FITC or Texas Red, as indicated) from Dianova, Hamburg. Phycoerythrin(PE)-conjugated streptavidin was obtained from Immunotech, Marseilles, France. Oligonucleotides were obtained from MWG, Ebersberg; vitronectin came from Sigma, Deisenhofen/Taufkirchen.

(B) cDNA-cloning and Mutagenesis

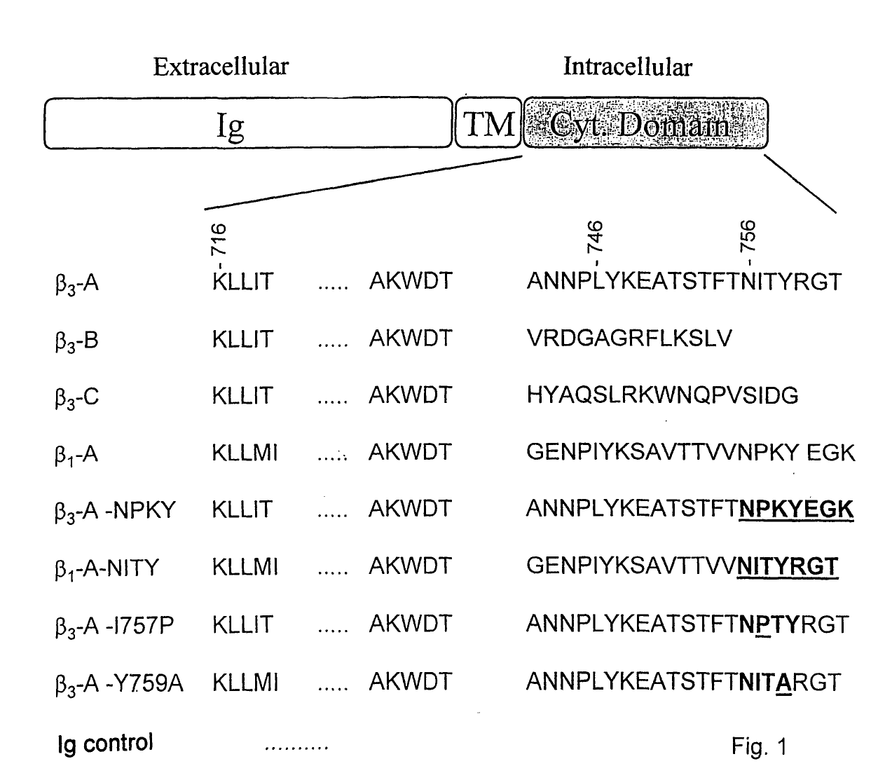

P5C7, a derivative of pRK5 containing a modified polylinker region, was used as the mammalian expression vector. Cytoplasmic Ig fusion proteins and transmembrane-CD16/CD17 chimeras were produced in the manner recently described (Kolanus, W. et al., Cell 86 (1996), 233-242). The transmembrane-Ig fusion proteins comprise the leader sequence of CD5, extracellular CH2 and CH3 domains of human IgG₁, the CD7 transmembrane domain and the intracellular tail of β3-integrin of complete length as indicated (FIG. 1). An Ig construct lacking the cytoplasmic domain served as a control (Ig control). The cytoplasmic domains of β1-A and β3-A were amplified by means of PCR of wild-type β1-A- and β3-cDNA sequences using primers having the corresponding coding sequences and restriction sites at the start and end of the open reading frame. After cleavage with MluI and NotI, the corresponding PCR products were cloned in P5C7. The β3-B and β3-C constructs were produced in an analogous manner. The β3-A variants V-A-NPKY, β3-A-Y759A, β3-A-1757P and the β1-A variant β1-A-NITY were likewise produced by means of PCR.

The cytoplasmic domains of the β3-integrins and their mutations were cloned via amplification by means of PCR, using the following oligonucleotides: β3-A: 5′-GGG GCG ACG CGT AAA CTC CTC ATC ACC ATC CAC-3′ (forwards)/5′-GGG GCG GCG GCC GCT TTA AGT GCC CCG gTA CGT GAT ATT GGT GAA-3 (backwards)'; β3-1757P: 5′-GGG GCG GCG GCG GCT TTA AGT GCG CCG GTA CGT ggg ATT GGT GAA GGT AG 3 (backwards)'; β3-Y759A: 5′-GGG GCG GCG GCG GCT TTA AGT GCC CCG GGC CGT GAT ATT GGT GAA GGT AG-3 (backwards)'; β3-NPKYEGK: 5′-GGG gCG GCG GCC GCT TTA TTT TCC CTC ATA CTT CGG ATT GGT GAA GGT AGA CGT GGC CTC-3′ (backwards); β1-NITYRGT: 5′-Ggg GCG GCG GCC GCT TTA AGT GCG CCG GTA CGT GAT ATT GAC CAC AGT TGT TAC GGC ACT-3′ (backwards).

Both β3-endonexin isoforms were cloned from a natural killer cell cDNA bank (Clontech, Heidelberg) via amplification by means of PCR, using the following oligonucleotides: 5′-GGG GCG ACG CGT ATG ATG GCT GTT AAA AGA TCA CTG AAG TTG GAT GGT CTG-3′ (forwards)/5′-GGG GCG GCG GCC GCT TCA CAG AGG TTG TGA CAT CTG AGG CTG AGG CTG ACC TTT GTG-3′ (backwards) (β3-endonexin-long) or 5′-GGG GCG ACG CGT ATG ATG CCT GTT AAA AGA TCA CTG AAG TTG GAT GGT CTG-3′ (forwards)/5′-GGG GCG GCG GCC GCT TCA CTG TAT ACT ACT TAA ATT TTG CAT TAT CTC CAT-3′ (backwards) (β3-endonexin-short). The resulting PCR fragments were fused with the corresponding 3-termini of an eGFP cloning cassette (Clontech, Heidelberg). β3-Endonexin mutants with deletions of the putative nuclear import sequence K⁶²RKK were produced by means of a two-step PCR strategy. All the constructs were identified by means of restriction cleavage, purified by CsCl centrifugation and verified by means of DNA sequencing prior to transfection.

β3-Endonexins were likewise cloned as constructs without eGFP into the same expression vector.

(C) Cell Lines and Transient Infections

CHO cells came from the American Type Culture Collection (ATCC, Rockville, Md.) and were kept in Dulbecco's modified Eagle medium (DMEM, Sigma) that had been supplemented with 10% foetal calf serum (FCS), 1% L-glutamine, 1% penicillin and streptomycin and 1% non-essential amino acids. Stable CHO cell lines expressing α_{IIb}β3 and α_{IIb}β3 (Y759A) have already been characterised before (Ylänne, J., et al., J. Biol. Chem. 270 (1995), 9550-9557). The cell lines were cultivated in DMEM supplemented with 0.75 mg/ml of geneticin (G418) disulfate.

Human umbilical endothelial cells (HUVEC) were purchased from Clonetics, California, USA. They were cultivated in “Endothelial Basal Medium” (from the same manufacturer) and passaged 5×maximum.

cDNA constructs were expressed in CHO cells by means of liposome-mediated transfection (Superfect™, Quiagen, Hilden). 24 hours before transfection, the cells were plated onto culture plates having six wells. A total of 2 μg of each construct and 10 μg of Superfect™ (Quiagen) were incubated at room temperature for 10 minutes in 110 μl of non-supplemented DMEM or M-199 (Gibco Life, Karlsruhe). Then, 600 μl of supplemented medium were added thereto and the DNA-Superfect™ complexes were laid over the cells. The cells were incubated for 2 hours at 37° C., washed with PBS and then incubated with complete medium at 37° C. After 24 hours, the medium was changed and analysis of the cells was carried out at 48 hours.

(D) Flow Cytometry and Confocal Laser Immunofluorescence Microscopy

Transient transfectants were harvested in Tyrode buffer containing 0.1 mg/ml of trypsin treated with L-1-tosylamido-2-phenylethylchloromethyl ketone (Sigma) and 3.5 mM EDTA. After incubation for 5 minutes at room temperature, the cells were diluted with Tyrode buffer containing 0.1% soybean trypsin inhibitor (Sigma) and 10% bovine serum albumin (BSA) and were fixed for 30 minutes at room temperature by the addition of an equal volume of 2% formaldehyde in PBS. The cells were then recovered by centrifugation at 1200 rev/min (5 min.) and washed once in Tyrode buffer. For the surface IgG staining, the fixed cells were resuspended in 100 μl of Tyrode buffer containing an excess of phycoerythrin(PE)-conjugated mouse-anti-IgG₁-MAK (50 μg/ml). After incubation for one hour, the cells were washed twice with 2% glycine in PBS and, for the intracellular Ig staining, were resuspended once more in Tyrode buffer containing fluorescein(FITC)-conjugated mouse-anti-IgG₁-MAK (50 μg/ml) and 0.2% Triton X-100™ for permeabilisation of the fixed cells. After incubation for a further hour, the cells were again washed twice, resuspended in 0.5 ml of Tyrode buffer and analysed by means of flow cytometry on a “FACScan” (Becton Dickinson, Heidelberg). 10,000 green-fluorescent transfectants (positive for intracellular FITC-anti-IgG staining) were analysed with regard to red immunofluorescence (PE) (surface-PE-anti-IgG staining). In experiments with GFP-labelled proteins, the fixed cells were surface-stained with PE-anti-IgG without permeabilisation. For the confocal laser immunofluorescence microscopy, transfected cells were incubated overnight at 4° C. on cover glasses coated with vitronectin (5 μg/ml) and blocked for 1 hour at room temperature with 5% BSA in PBS. Cell monolayers were then fixed and stained as described above for the flow cytometry analysis. The immunofluorescence analysis was carried out by means of a confocal laser microscope (Leica, Bensheim) equipped with appropriate software.

(E) Cell Lysis and Immunoprecipitation

For the immunoprecipitation, subconfluent monolayers of CHO cells were transiently transfected with Ig or GFP fusion constructs. Cells were lysed by the addition of lysis buffer containing 100 mM Tris, pH 8.0, 150 mM NaCl, 2 mM EDTA and 1% Triton X-100T™. After 20 minutes' incubation at 4° C., the lysates were centrifuged at 20,000×g to remove insoluble material. Ig fusion proteins were then recovered directly by means of protein A-6MB-sepharose beads (Amersham Pharmacia Biotech., Freiburg). Immunoprecipitates were washed three times with lysis buffer before dissociation in SDS application buffer. The proteins were separated on SDS-polyacrylamide gels and transferred to nitrocellulose membranes. Immunological detection was carried out by means of peroxidase-conjugated secondary antibodies (Dianova) and chemiluminescence (Amersham Pharmacia Biotech).

(F) Endocytosis

The endocytosis of β3-integrins was measured by means of a process that had been modified in accordance with Bretscher (1992) (Bretscher, M. S., EMBO. J. 11 (1992), 405-410). Similar levels of stable cells expressing α_{IIb}β3 and α_{IIb}β3 (Y759A) were cultivated overnight on plates having a diameter of 35 mm, washed twice with Dulbecco's modified PBS (Life Technologies, Karlsruhe), pre-incubated with biotin-labelled anti-β3-MAK (5 μg/ml) on ice for 15 minutes and incubated in Dulbecco's modified PBS at 37° C. for a further 0, 2, 5, 15, 30 or 60 minutes. The cells were then washed twice, followed by three successive 5-minute incubations with a reducing solution containing 50 mM 2-mercaptoethanesulfonic acid (Sigma), 10 mM NaCl, 1 mM EDTA, 50 mM Tris and 0.2% bovine serum albumin at a pH of 8.6. The cells were detached, and an aliquot thereof was fixed with 2% formaldehyde for 30 minutes on ice, washed twice with 2% glycine and incubated for a further 30 minutes with PE-streptavidin in the presence of 0.2% Triton-X 100™. The cells were then analysed by means of flow cytometry, and the mean intensity of PE-streptavidin of 10,000 events was used as a measure of the β3-integrin endocytosis. A further aliquot of CHO cells was incubated for the time indicated with biotin-conjugated anti-β3-MAK, after reduction with 2-mercaptoethanesulfonic acid was lysed at 4° C. with 200 μl of 200 mM n-octyl-β-D-glucopyranoside (Sigma), 1 mM phenylmethylsulfonyl fluoride (Sigma) in Dulbecco's modified PBS and centrifuged at 12,000×g for 5 minutes. Then, the same volume of 100 mM Tris, 150 mM NaCl, 1 mM CaCl₂, 1% Triton X-100™, 0.1% SDS and 0.1% Nonidet P-40, pH 7.4, was added. Finally, 20 μg of protein per lane were applied to 8% acrylamide flat gels and, after separation under non-reducing conditions, the proteins were transferred to an Immobilon™ membrane (Millipore, Eschbom) using 25 mM Tris, 192 mM glycine, 20% methanol, pH 8.3 value (Ylänne, J., et al. supra). Biotin-labelled anti-β3-MAKs were visualised by means of 2 μg/ml of peroxidase-coupled streptavidin (Dianova) and a chemiluminescence immunoblot kit (Roche Diagnostics, Mannheim).

Gel Shift Assays for Measuring the NF-kB Activity:

Nuclear extracts were prepared from HUVEC and analysed as described. The prototypic immunoglobulin-kappa-chain-oligonucleotide was used as a probe and was labelled with Klenow fragment DNA polymerase I (Roche, Mannheim). Nuclear proteins were incubated in 20 μl of binding buffer with ³²P-dCTP for 30 minutes at 25° C. The samples were separated in 0.25% TBE (TRIS, borate, EDTA, pH 8.0) on non-denaturing 4% polyacrylamide gels. The gels were then dried and autoradiographed.

(G) Transfections

Reporter plasmids were transfected with 3×10⁵ CHO cells using the SuperFect™ transfection reagent (Qiagen, Hilden, Germany) and inoculated overnight into plates having 6 wells. For the chloramphenicol acetyl transferase (CAT) assays, all the transient transfections were carried out in the presence of 1 μg of u-PAR-CAT reporter constructs and 1 μg of luciferase expression vector (see next paragraph) and, where indicated, in addition with 3 μg of expression plasmid coding for β3-integrin or with equimolar amounts of the control vector. After three hours, the cells were rinsed twice with PBS, then changed to medium containing 10% FCS and grown for a further 45 hours. The cells were harvested and then lysed by repeated freezing/thawing cycles in 0.25 M Tris-HCl, pH value 7.8. The transfection efficiency was determined by means of luciferase activity assays. After normalisation for the transfection efficiency, the CAT activity was measured by incubation of the cell lysates with 4 μM [¹⁴C]-chloramphenicol and 1 mg/ml of actely coenzyme A at 37° C. The mixture was separated off by extraction with ethyl acetate and the acetylated products were separated on thin-layer chromatography plates using chloroform/methanol as the mobile phase. The reaction products were visualised by means of autoradiography and the radioactivity was quantified by means of a 445 SI Phosphor Imager (Molecular Dynamics).

For luciferase assays, the transient transfections were carried out in the presence of 1 μg of u-PAR-luciferase reporter construct and 10 ng of a Renilla-luciferase plasmid (pRL-SV40; Promege, Mannheim) as the internal control together with 3 μg of β3-integrin-coding expression plasmid or the control vector. After transfection, the cells were grown in serum-free medium for three hours and then, in addition, with 10% FCS for a further 45 hours. Firefly luciferase activity was standardised for Renilla luciferase which was used as the internal control.

### EXAMPLE 2

### Isoform-dependent Differential Cell Surface Expression of β3-integrin Chimeras

β3-Integrins are involved in important biological processes, it being assumed that the cytoplasmic domains of the β sub-units play a role, for example, in the regulation of the extracellular binding of ligands (“inside-out signalling”), signal transduction and internalisation/recycling of the receptors. To understand the functional significance of the three known cytoplasmic β3 domain isoforms, single-chained chimeric receptors carrying CH2 and CH3 portions of human IgG1 at the cell surface, the transmembrane domains of CD7 and the cytoplasmic domains β3-A, -B and -C of complete length (FIG. 1) were therefore produced. An analogous construct carrying the cytoplasmic domain of β1-A or a tailless Ig construct were used as further controls. All of the constructs were based on recently described chimeric receptors (Kolanus, W., et al., Cell 74 (1993) 171-183). On expression of these chimeras in various cell systems, it was found that the β3-A chimeras exhibited a drastically reduced cell surface expression in comparison with other β3 isoform fusion proteins or control chimeras. CHO cells (FIG. 2A) were transiently transfected with Ig chimeras and the cells were stained sequentially with fluorescein- or Texas-Red-coupled anti-Ig antibodies for detection of both the cell surface fraction and the intracellular expression of the fusion proteins. As shown in FIG. 2A, Ig-β3-B, Ig-β3-C and Ig-β1-A exhibited a diffuse intracellular and “steady state” plasma membrane surface staining pattern that resembled the staining pattern of the tailless Ig chimeras. In contrast, Ig-β3-A was primarily intracellularly localised (FIG. 2A). For a more quantitative assessment of the surface expression of the fusion proteins, CHO cells were transiently transfected with the corresponding constructs, fixed and, having been double-stained, were examined with regard to intracellular and surface expression by means of flow cytometry. As shown in FIG. 2B, the surface expression of Ig-β3-A was significantly reduced in comparison with the mutants having alternative splicing sites (Ig-β3-B, Ig-β3-C), the Ig-β1 chimeras or the tailless Ig construct. The differences in the surface expression of the tested chimeras were not due to differences in the total protein concentration, since the average intensity of the intracellular immunofluorescence was identical for all the transfected constructs, and immunoblot analysis confirmed that the “steady state” expression levels of all the chimeras tested were very similar (FIG. 2C). These results imply, therefore, that the cytoplasmic β3-A domain is involved in the regulation of the cell surface expression of this particular β3-integrin.

### EXAMPLE 3

### The NITY Motif in β3-A 756-759 is Critical for the Surface Expression of the Ig-β3 Chimeras

The cytoplasmic domains of the β3-integrins interact with a number of proteins of the cytoskeleton and signal proteins. Conserved regions, such as, for example, NPXY and NXXY which are present in most cytoplasmic β domains, mediate the localisation of the β3-integrins inside the plasma membrane during spreading of the cells and the formation of heart-shaped adhesions. The question was, therefore, whether mutations or deletions of the C-terminal NITY motif, which is expressed exclusively in the cytoplasmic β3-A sequence (FIG. 1), influence the surface expression of the corresponding chimeras. In fact, it was found that substitution of the carboxy terminus of β1 by the C-terminal seven residues of the cytoplasmic β3-domain (756-762) (NITYRGT, FIG. 1) or, alternatively, substitution of the NITYRGT motif in β3-A by the NPKYEGK motif (772-778) of β1-A (FIG. 1) led to considerably increased surface expression of the Ig-β3-A-NPKY mutant and a simultaneous decrease in the surface detection of the Ig-β1-A-NITY chimera (FIGS. 3A and 3B). In addition, the introduction of Pro⁷⁷³ of β1-A into the cytoplasmic β3-A domain (β3-A-1757P) increased the surface expression of the chimeras considerably (FIGS. 3A and 3B). Similar results with increased cell surface expression were obtained in β3-A with an alanine substitution at Tyr⁷⁵⁹ (β3-A-Y759A) (FIGS. 3A and 3B). These data show that the linear sequence motif of β3-A N⁷⁵⁶ITY is of critical importance for a reduced cell surface exposition of the β3-A-β3-integrin fusion protein. The N⁷⁵⁶ITY motif is also required for the “inside-out” signal transmission in blood platelets and CHO cells and mediates the interaction with the cytoplasmic protein β3-endonexin.

### EXAMPLE 4

### β3-Endonexin Regulates the Surface Expression of Ig-β3 Fusion Proteins

β3-Endonexin binds to the cytoplasmic domain of the β3-A sub-unit. That interaction is specific, since it has not been observed with the cytoplasmic domains of other β3-integrins. It was furthermore possible to show that the binding of β3-endonexin to β3-A is dependent on the N⁷⁵⁶ITY motif which is present only in β3-A (FIG. 1). Two isoforms of β3-endonexin were described which differ in size and, more significantly, in their capacity to bind to the cytoplasmic domain of β3-A (FIG. 4A). The “short” β3-endonexin splice variant (β3-endonexin-S; En-S) consists of 111 amino acids and binds to β3-A, whereas the longer form of β3-endonexin (170 amino acids, β3-endonexin-L; En-L) does not interact with the β3-A tail (see the diagram in FIG. 4A). Since the N-termini of both polypeptide variants are identical, those two isoforms were used to investigate whether β3-endonexin is involved in the regulation of the cell surface expression of β3-A. To investigate the effect of both isoforms of β3-endonexins on the cell surface expression of β3-A, cDNAs for both isoforms were isolated from a natural killer cell line cDNA bank and fused for customary sub-cellular detection to the C-terminus of the green fluorescent protein (GFP). Since GFP/β3-endonexin is expressed both in the cytoplasm and in the nucleus, both mutants of the two β3-endonexin isoforms that are constitutively localised in the cytoplasm, that is to say, in which the putative signal for nuclear localisation at position K⁶²RKK had been deleted, were constructed. As shown in FIG. 4C, with regard to the nuclear import of both β3-endonexin isoforms (En-SΔK⁶²RKK and En-LSΔK⁶²RKK), mutants exhibited a considerably greater degree of cytoplasmic GFP-fluorescence in comparison with the wild-type β3-endonexin chimeras. Upon co-transfection of the wild-type and the mutated β3-endonexin isoforms (FIG. 4C) with Ig-β3-integrin chimeras in CHO cells or HUVEC, it was found that the surface expression of Ig-β3-A in cells co-transformed with the long form of β3-endonexin was markedly increased in comparison with cells co-transformed with the short form of β3-endonexin or with control constructs (FIGS. 4C and 4D). The surface expression of β3-A was increased to a slightly greater degree if the cells had been co-transfected with the nuclear import mutant of the long form of β3-endonexin (En-LΔK⁶²RKK). The short-form mutant (En-SΔK⁶²RKK) did not show any significant effect on the cell surface expression of Ig-β3-A (FIGS. 4C and 4D). Upon co-transfection of GFP/β3-endonexin and the Ig-β3-A-NITY chimeras, it was similarly observed that En-L significantly increases the surface expression of that fusion protein (FIGS. 4C and 4D), whereas no substantial effect of the two β3-endonexin isoforms on the surface expression of the other Ig-β3-integrin chimeras was observed (FIGS. 4C and 4D). These findings clearly suggest that the β3-endonexin system specifically regulates the cell surface expression of β3-A. The fact that both GFP/β3-endonexins are localised primarily in the nucleus does not appear to play any significant role in this connection: although the constitutive cytoplasmic localisation of GFP-β3-endonexin-L increases the plasma membrane expression of β3-A to a slightly better degree than that of the wild-type version, both constructs exhibit qualitatively similarly effects.

### EXAMPLE 5

### β3-A-integrin Fusion Proteins are Internalised Via a β3-endonexin-dependent Mechanism

It is known that β3-integrins are internalised via endocytosis, the cytoplasmic domains playing an important role in that process. To assess the role of the cytoplasmic domains of β3-integrin in the endocytotic mechanism, CHO cells were transiently transfected with Ig-β3-integrin chimeras. After 24 hours, a fluorochrome-labelled anti-Ig-MAK was added to the living cells for 15 minutes. The antibody was then removed and the cells were fixed with 2% formaldehyde at the times indicated. It was found that the anti-Ig antibody was rapidly internalised in β3-A-transfected cells and, within minutes, could be detected in a vesicular compartment beneath the plasma membrane (FIG. 5A). A longer incubation period led to a centralisation of the anti-Ig-FITC immunofluorescence. Similar results were found with Ig-β1 constructs that contained the N⁷⁵⁶ITY motif of β3-A. In contrast, a predominant or exclusive surface staining with virtually no or only minimal cellular absorption of anti-Ig-MAK was observed in cells that had been transiently transfected with the other isoforms β3-B or β3-C or the β1-A or tailless Ig chimeras (FIG. 5A). These results show that, although the “steady state” surface expression of β3-A is greatly reduced (FIGS. 2A and 2B), a proportion of the cellular pool of the Ig-β3-A fusion proteins obviously cycles between the plasma membrane and an intracellular vesicular compartment. In contrast, cells that had been transfected with point mutants of β3-A, β3-A-Y759A and β3-A-1757P or with other β3 isoforms, which exhibited a considerable “steady state” surface expression (FIGS. 2 and 3), exhibited a considerably lower internalisation of the anti-Ig antibody (FIG. 5A). It can therefore be concluded that the N⁷⁵⁶ITY motif is involved in the internalisation or endocytosis of β3-A-integrin.

It was then investigated whether β3-endonexin modulates the internalisation of β3-A. For this, CHO cells were co-transfected with the β3-endonexin isoform-GFP fusion proteins or mutants thereof and an Ig-β3 chimera. The absorption of a FITC-conjugated anti-Ig-MAK was monitored by means of immunofluorescence microscopy at the intervals indicated. It was found that, in cells that had been co-transfected with the chimeras of the long isoform and Igβ3-A, the absorption of the FITC-anti-Ig-MAK was considerably less pronounced in comparison with cells co-transfected with En-S constructs (FIG. 5B). These results show that the internalisation of anti-Ig-MAK-ligated β3-A integrins is regulated by the cytoplasmic β3-A-binding protein β3-endonexin.

### EXAMPLE 6

### Anti-β3-MAK-induced Endocytosis of Native β3-integrins is Regulated Via a β3-endonexin-dependent Mechanism

In order to investigate whether β3-endonexin is involved in the internalisation of the native β3-integrin, cell lines expressing wild-type α_{IIb}β3 and α_{IIb}β3(Y759A) were incubated with a biotin-labelled anti-β3-MAK either on ice or at 37° C. at the intervals indicated and subjected to a membrane-impermeable reducing agent. That compound abolished the streptavidin-FITC-mediated detection of the antibody and therefore provided a means of measuring the internalisation of the receptor.

FIG. 6A demonstrates that wild-type and mutant β3-integrins were expressed in similar density on the surface of CHO cells. Subsequent immunoblot experiments showed that approximately 40% of the biotin-labelled anti-β3 antibody that was bound to the cell surface at 4° C. were protected from reduction if cells expressing wild-type α_{IIb}β3 were used (FIGS. 6B and 6C). In cells expressing α_{IIb}β3(Y⁷⁵⁹A), however, only 5-6% of the material bound at low temperature were protected from reduction (FIGS. 6B and 6C), which indicates that considerable endocytosis took place in the wild-type but not in cells expressing mutant β3. Accordingly, as described above for the β3-integrin chimeras, the cytoplasmic domains of native β3-integrin and the NITY⁷⁵⁹ sequence are required for significant endocytosis of α_{IIb}β3 in CHO cells.

Analogous experiments were then carried out using immunofluorescence methods. Microscopic analyses showed that considerable amounts of a surface-bound, biotin-labelled MAK were transferred to intracellular sites in cells expressing wild-type α_{IIb}β3 (FIG. 7B, left part). In contrast, the internalisation of the biotin-labelled anti-β3-MAK in cells expressing α_{IIb}β3(Y759A) was significantly reduced (FIG. 7B, right part). In the mutant (FIG. 7B, right part), the antibody used for detection was never detectable in large vesicular structures as was to be observed in cells expressing wild-type β3. Any surface-bound antibody that was protected remained in any case associated with the cell cortex.

In order to obtain a more quantitative assessment of the β3-integrin-dependent internalisation measured by means of immunofluorescence, the cells were examined by flow cytometry. As illustrated in FIG. 7A, the absorption of the PE-streptavidin fluorescence in cells expressing α_{IIb}β3(Y759A) (circles) was significantly reduced in comparison with cells expressing wild-type α_{IIb}β3 (squares).

### EXAMPLE 7

### The Transient Over-expression of GFP-endonexin-L Inhibits the Endocytosis of α_{IIb}β3 in CHO Cells

In order to assess whether the over-expression of β3-endonexin also modulates the endocytosis of the native β3-integrin, cells expressing α_{IIb}β3 or α_{IIb}β3(Y759A) were transiently transfected with various GFP/β3-endonexin fusion constructs and incubated with biotin-labelled anti-β3-MAK for 15 minutes at 37° C. After reduction with 2-mercaptoethanesulfonic acid, the cells were stained with PE-streptavidin and examined by means of flow cytometry. In agreement with the experiments in which β3-A chimeras were used, it was found that the expression of the long form of β3-endonexin or the deletion mutant En-LΔK⁶²RKK led to a significant, dosage-dependent inhibition of PE-streptavidin fluorescence absorption, whereas En-S showed no effect. These results show that endocytosis of the native β3-integrin is regulated by the cytoplasmic β3-endonexin (FIG. 8).

### EXAMPLE 8

### β3-Integrin Internalisation in Storage Vesicles (α-granules) of Thrombocytes from Patients with Coronary Heart Disease

In order to investigate to what extent β3-integrins on human thrombocytes (GPIIb-IIIa) are internalised in α-granules, thrombocyte-rich plasma was separated from the blood of patients with coronary heart disease, anticoagulated with acidic citrate-dextrose (ACD; according to formula A of the National Institute of Health, Bethesda, Md., USA), by centrifugation at 230×g for 16 minutes. The thrombocytes were then washed and resuspended in a medium containing 90 mM NaCl, 5 mM KCl, 2 mM CaCl₂, 1 mM MgCl₂, 5 mM glucose, 30 mM phosphate buffer pH 6.5. The thrombocytes were incubated with 20 μg/ml of 7E3 monoclonal antibody (inhibits the fibrinogen binding site of GP IIb/IIIa and was supplied by Dr. Coller, New York, USA) for 5 minutes at 20° C. As a negative control, incubation was carried out with 20 μg/ml of non-specific mouse IgG. After centrifugation once more (5 min., 180×g) and resuspension in 120 mM NaCl, 5 mM KCl, 1 mM CaCl₂, 0.1 mM MgCl₂, 5 mM glucose, 1 mM phosphate buffer and 30 mM TES buffer at pH 7.4, 10 μg/ml of 5 nm-gold-labelled anti-mouse-IgG Fab fragments (Biotrend, Cologne) were added. The suspension was incubated for 10, 20, 60 and 90 minutes. The immunolabelled samples were then fixed with 0.2% glutaraldehyde and prepared as described for electron microscopy.

The time progression illustrated in FIGS. 9A-C shows that thrombocyte α-granules are substantially sheathed by glycoproteins IIb/IIIa, the β3-integrin receptors of the thrombocyte, internalised from the cell surface. It becomes clear from this that inhibition of β3-integrin-inhibition has a substantial effect on the composition and the function of the storage vesicles.

### EXAMPLE 9

### β3-Integrin Internalisation in α-granules Causes Reduced α-degranulation

In order to be able to assess, on the basis of P-selectin surface expression on human thrombocytes, the degranulatory activity thereof and hence the release of vasoactive and atherogenic mediators from α-granules, thrombocyte-rich plasmas from the mentioned whole blood were enriched by centrifugation (200×g for 20 minutes). The final thrombocyte count was adjusted to 2×10⁸/ml by means of autologous plasma. After setting the zero values, 5 μl of TRAP (25 μM) were added; the P-selectin surface expression was determined by means of flow cytometry (described in: Gawaz M., et al., Thromb Haemost 80 (1998), 994-1001). A monoclonal anti-CD62P antibody (Klan CLBThromb/6) produced by Immunotech, Marseilles, France, that specifically recognises P-selectin on the surface of degranulated thrombocytes was used for that purpose.

It was found (FIG. 10) that the P-selectin surface expression/secretion from thrombocytes (as an example of the secretion of various vasoactive mediators, such as serotonin, growth factors or cytokines) is dependent on the internalisation of their surface β3-integrins, the glycoprotein IIb/IIIa receptors, which occurs at the same time (FIG. 9).

An increased β3-integrin internalisation reduces time-dependently the secretion of vasoactive substances, since the mediator reuptake capacity of the α-granules falls. Accordingly, direct promotion of that internalisation process, such as by β3-endonexin-short over-expression/activation or endonexin-long inhibition, can markedly reduce that secretion.

### EXAMPLE 10

### β3-Endonexin-short Down-regulates the uPAR Promoter

3×10⁵ CHO cells were seeded overnight in 6-well dishes, then transiently transfected by means of SuperFect transfection reagent (Qiagen, Hilden). For the chloramphenicol acetyl transferase tests, 1 μg of a CAT reporter construct that was under the control of the urokinase-type plasminogen activator receptor (uPAR) promoter was used. For that purpose, a 449 bp fragment of the uPAR gene (sequence position −398 to +51) was cloned into the Xbal cleavage site of the pCAT basic vector (Promega, Mannheim). In addition, 1 μg of luciferase expression vector in each case and various amounts of β3-endonexin-short plasmid (200 ng to 5 μg) or the empty expression vector (pEGFP) in equimolar amount were co-transfected. After 3 hours, the cells were washed twice with PBS, transferred to medium containing 10% foetal calf serum (Gibco, Karlsruhe) and cultivated for a further 45 hours. The cells were then harvested and lysed by repeated freezing/thawing cycles in 0.25 M Tris-HCl, pH 7.8. Cell extracts that had been normalised with regard to luciferase activity were incubated with 4 μM ¹⁴C-chloramphenicol and 1 mg/ml of acetyl coenzyme, extracted with ethyl acetate and subjected to thin-layer chromatography using chloroform/methanol as the mobile phase. The conversion of ¹⁴C-chloramphenicol to acetylated derivatives was carried out by means of a phosphorimager (Molecular Dynamics, Heidelberg, type 445 SI). The results are illustrated in FIG. 11. Expression of endonexin-short inhibits markedly the activation of the uPAR promoter in a concentration-dependent manner. This shows that the expression of uPAR receptors depends concentration-dependently on the presence of β3-endonexin-short, which underlines the latter's importance in the process of cell migration and hence in plaque destabilisation.

### EXAMPLE 11

### Release of MCP-1 from Human Endothelial Cells

Cultivated HUVEC cells were transfected either with a plasmid construct coding for β3-endonexin-short or β3-endonexin-long as in Examples 5 and 6 or with an empty control vector. 24 hours later, the cells were incubated for 120 minutes either with vehicle or with 100 pg/ml of interleukin-1β. The supernatant was then removed by suction, centrifuged at 4000 rev/min for 10 minutes and stored at −80° C. The concentrations of monocyte chemotactic protein 1 (MCP-1) were determined by means of ELISA (R&D Systems, Wiesbaden, Order No. DCP00) with a detection limit of 5 μg/ml in accordance with the manufacturer's instructions. The results (FIG. 12) show that the over-expression of β3-endonexin-short lowers both the basal and the interleukin-induced release of the atherogenic MCP-1 and therefore can favourably influence atherosclerotic processes.

### EXAMPLE 12

### β3-Endonexin-long Increases the Density of Vitronectin Receptors and Reduces Their Internalisation Also in Human Endothelial Cells

The experiments of FIG. 13 show, analogously to the results in transfected CHO cells, also in human endothelial cells that the density of the vitronectin receptors increases markedly after over-expression of β3-endonexin-long. This clearly indicates that En-L is able to bring about a reduction in the internalisation of β3-integrins also on endothelial cells.

### EXAMPLE 13

### β3-Endonexin-short Interacts Directly with the Transcription Factor p65 of NF-kB and Thus Prevents Cytokine-induced Stimulation of NF-kB

The experiments of FIGS. 14 and 15 show that En-S and En-L interact directly with the nuclear transcription factor NF-kB. That binding reduces the activatability of that factor by cytokines, as can readily be seen by reference to the clear reduction in the NF-kB binding activity in the gel.

### EXAMPLE 14

### β3-Endonexin-long, Not β3-endonexin-short, is the Dominant Protein in Human Thrombocytes and Endothelial Cells

FIG. 16 shows that it is not β3-endonexin-short, as had hitherto been assumed, but β3-endonexin-long that is the dominant protein in human thrombocytes and endothelial cells. On the basis of the results on the transfected CHO cells it can be concluded that the internalisation of thrombocytic fibrinogen receptors is inhibited by that protein.

1. The use of β3-endonexin-long or β3-endonexin-short for finding active substances for the treatment of arteriosclerosis, unstable plaques resulting from the latter, acute coronary thrombosis, cardiac infarct, stroke, peripheral arterial occlusion diseases, chronic venous ulcer and restenosing processes.

2. A method of identifying compounds that inhibit binding of β3-endonexin-short to β3-integrin, characterised by the following steps:
(a) incubation of a mixture comprising
(a1) β3-endonexin-short or a peptide or polypeptide that is functionally equivalent thereto with regard to binding to β3-integrin;
(a2) β3-integrin or a peptide or polypeptide that is functionally equivalent thereto with regard to binding to β3-endonexin-short;
(a3) a compound to be tested; and
(b) detection of the inhibition of the binding of component (a1) to component (a2) in the presence of compound (a3) in comparison with the absence of compound (a3).

3. A method of identifying compounds that inhibit an influence of β3-endonexin-long on β3-integrin, characterised by the following steps:
(a) incubation of a mixture comprising
(a1) β3-endonexin-long or a peptide or polypeptide that is functionally equivalent thereto with regard to binding to β3-integrin;
(a2) β3-integrin or a peptide or polypeptide that is functionally equivalent thereto with regard to binding to β3-endonexin-long;
(a3) a compound to be tested; and
(b) detection of the inhibition of an influence of component (a1) on component (a2) in the presence of compound (a3) in comparison with the absence of compound (a3).

4. A method of identifying compounds that shift the competition of the binding of β3-integrin to β3-endonexin-short and β3-endonexin-long, characterised by the following steps:
(a) incubation of a mixture comprising
(a1) β3-endonexin-short or a peptide or polypeptide that is functionally equivalent thereto with regard to binding to β3-integrin;
(a2) β3-endonexin-long or a peptide or polypeptide that is functionally equivalent thereto with regard to binding to β3-integrin;
(a3) β3-integrin or a peptide or polypeptide that is functionally equivalent thereto with regard to binding to β3-endonexin-short and/or to β3-endonexin-long;
(a4) a compound to be tested; and
(b) detection of a shift in the ratio of the binding of component (a1) to component (a3) to the binding of component (a2) to component (a3) in comparison with the absence of the compound to be tested (a4).

5. A test mixture for identifying compounds that inhibit binding of β3-endonexin-short or β3-endonexin-long to β3-integrin, characterised by the following components:
(a) a first component selected from the group consisting of β3-integrin and peptide or polypeptide that is functionally equivalent thereto with regard to binding to β3-endonexin-short and/or to β3-endonexin-long;
(b) a second component selected from a first and/or a second group, wherein
(b1) the first group consists of β3-endonexin-short and peptides or polypeptides that are functionally equivalent thereto with regard to binding to β3-integrin;
(b2) the second group consists of β3-endonexin-long and peptides or polypeptides that are functionally equivalent thereto with regard to binding to β3-integrin.

6. A kit for identifying compounds that inhibit binding of β3-endonexin-short or β3-endonexin-long to β3-integrin, characterised by the following components:
(a) a first component selected from the group consisting of β3-integrin and peptide or polypeptide that is functionally equivalent thereto with regard to binding to β3-endonexin-short and/or to β3-endonexin-long;
(b) a second component selected from a first and/or a second group, wherein
(b1) the first group consists of β3-endonexin-short and peptides or polypeptides that are functionally equivalent thereto with regard to binding to β3-integrin;
(b2) the second group consists of β3-endonexin-long and peptides or polypeptides that are functionally equivalent thereto with regard to binding to β3-integrin.

7. A compound obtainable or obtained by any one of the methods of claim 2, 3 or 4.

8. A medicament that comprises a compound for influencing β3-integrin-dependent intracellular process, the compound being characterised in that, for promoting β3-integrin-internalisation-dependent processes and/or for inhibiting the secretion of pro-atherogenic mediators from endothelial cells and for inhibiting the expression of migration-promoting surface receptors on endothelial cells, it has an activating effect on β3-endonexin-short and/or an inhibiting effect on β3-endonexin-long, the compound being selected from
(a) (I) β3-endonexin-short or (II) an expression vector containing DNA coding for β3-endonexin-short;
(b) a ribozyme or a vector coding therefor, characterised in that it can bind specifically to β3-endonexin-long mRNA and cleave the latter, but cannot bind to β3-endonexin-short mRNA, whereby the synthesis of β3-endonexin-long is specifically reduced or inhibited;
(c) an antisense RNA or a vector coding therefor, characterised in that it can bind specifically to β3-endonexin-long mRNA, but not to β3-endonexin-short mRNA, whereby the synthesis of β3-endonexin-long is specifically reduced or inhibited;
(d) an anti-β3-endonexin-long antibody; and
(e) a compound according to claim 7.

9. A medicament that comprises a compound for influencing β3-integrin-dependent intracellular processes, the compound being characterised in that, for inhibiting β3-integrin-internalisation-dependent processes and/or for inhibiting the secretion of pro-atherogenic mediators from thrombocytes, it has an activating effect on β3-endonexin-long and/or an inhibiting effect on β3-endonexin-short, the compound being selected from
(a) (I) β3-endonexin-long or (II) an expression vector containing DNA coding for β3-endonexin;
(b) a peptide or polypeptide containing the NITY motif;
(c) a compound that inhibits the binding of β3-endonexin-short to the NITY motif of β3-integrin; and
(d) a compound according to claim 7.

10. The use of a compound defined in claim 7, 8 or 9 for the treatment of conditions that are associated with reduced activity of β3-integrin-internalisation-dependent processes and/or with increased secretion of pro-atherogenic mediators from endothelial cells and increased expression of migration-promoting surface receptors on endothelial cells or that can be treated by promoting β3-integrin-internalisation-dependent processes and/or inhibition of the secretion of pro-atherogenic mediators from endothelial cells and inhibition of the expression of migration-promoting surface receptors on endothelial cells.

11. The use according to claim 10, wherein the conditions are acute and chronic vascular diseases.

12. The use of a compound defined in claim 9 for the treatment of conditions that are associated with increased activity of β3-integrin-internalisation-dependent processes and/or increased secretion of pro-atherogenic mediators from thrombocytes or that can be treated by inhibition of β3-integrin-internalisation-dependent processes and/or inhibition of the secretion of pro-atherogenic mediators from thrombocytes.

13. The use according to claim 12, wherein the conditions are acute and chronic vascular diseases.

14. The use according to claim 11 or 13, wherein the vascular diseases are arteriosclerosis, unstable plaques resulting from the latter, acute coronary thrombosis, cardiac infarct, stroke, peripheral arterial occlusion disease, chronic venous ulcer and restenosing processes.

15. A method of treating acute or chronic vascular diseases, such as atherosclerosis and unstable vascular plaques, acute cardiac infarct, stroke, peripheral arterial occlusion disease, chronic venous ulcer of the extremities, restenoses following surgical intervention, wherein the method comprises administering a medicament according to either of claims 7 and 8.
